(19) United States
(12) Patent Application Publication
(10) Pub. No.: US 2026/0363859 A1
(43) Pub. Date: Nov. 19, 2026

(54) SUTURE MANIFOLD AND ADJUSTABLE SUTURE NET FOR ATRIOVENTRICULAR VALVE REPAIR

(71) Applicant: Jian YE, Vancouver (CA)

(72) Inventor: Jian YE, Vancouver (CA)

(21) Appl. No.: 19/682,273

(22) Filed: May 19, 2026

Related U.S. Application Data
(60) Provisional application No. 63/808,351, filed on May 19, 2025.

(51) Int. Cl. A61F 2/24; A61B 17/04

(52) U.S. Cl. CPC A61F 2/2457; A61B 17/0401; A61F 2/2445; A61F 2/2466; A61B 2017/0406; A61B 2017/0414; A61B 2017/0464; A61F 2220/0075; A61F 2230/0065

(57) ABSTRACT

Devices, systems, and methods can be used to repair atrioventricular heart valves, including the mitral valve and the tricuspid valve, through surgical and transcatheter approaches. A suture manifold can include a body, one or more inlets, one or more internal passages, and a plurality of outlets. The suture manifold can direct, organize, and control the travel of two or more sutures simultaneously. The internal passages of the suture manifold may be siloed from one another by one or more internal walls to separate distinct functional groups of sutures. An adjustable suture net is further provided, formed by the two or more sutures and one or more horizontal threads connecting them, wherein the tautness of the net is controlled by adjusting the length of each suture through proximal retraction or distal advancement.

### CROSS-REFERENCE TO RELATED APPLICATIONS

This application claims priority under 35 U.S.C. 119(e) to, and the benefit of, U.S. Provisional Application No. 63/808,351, filed May 19, 2025, the entirety of which is incorporated herein by reference.

### BACKGROUND

### Field of the Inventions

The present disclosure generally relates to the field of cardiac surgery and interventional cardiology, and in particular to devices and methods for the repair of atrioventricular heart valves, including the mitral valve and the tricuspid valve. Such repair procedures may be performed through an open surgical approach, in which the chest is opened and the heart is accessed directly under cardiopulmonary bypass, or through a transcatheter approach, in which devices are delivered through the vasculature without the need for open surgical access.

### Description of the Related Art

The mitral valve of the heart, also known as the bicuspid valve, is located between the left atrium and the left ventricle and controls the unidirectional flow of blood from the atrium into the ventricle during diastole. The tricuspid valve performs the analogous function on the right side of the heart, controlling flow between the right atrium and the right ventricle. Each of these valves comprises an annulus, which is a fibrous ring (mitral annulus) or muscular ring with some fibrous tissue (tricuspid annulus) at the base of the valve providing structural support; two or more leaflets, which are thin, flexible tissue flaps that open and close with each cardiac cycle; and a subvalvular apparatus, comprising the chordae tendineae and papillary muscles, which tether the leaflets to the ventricular wall and prevent them from prolapsing into the atrium during systole.

When any part of the valve apparatus becomes diseased or deteriorates over time, the valve may fail to close properly-a condition known as valvular insufficiency or regurgitation-allowing blood to flow backward from the ventricle into the atrium instead of forward through the heart. Mitral regurgitation is one of the most common forms of valvular heart disease, affecting millions of patients worldwide, and may result from leaflet prolapse, elongated or ruptured chordae tendineae, enlargement of the valve annulus, and leaflet thickening, among other possible causes. The tricuspid valve is susceptible to similar problems, most commonly as a downstream consequence of left-sided heart disease or elevated pulmonary pressure. Left untreated, significant valvular regurgitation forces the heart to work harder to compensate for the backward flow of blood, leading over time to progressive cardiac deterioration, heart failure, and reduced survival.

Surgical repair of the mitral and tricuspid valves has been practiced for decades and encompasses a range of techniques. Annuloplasty, in which a prosthetic ring or band is sutured to the valve annulus to reduce annular dilation and restore annular geometry, is the most common procedure. Chordal repair techniques, including chordal shortening, chordal transposition, and artificial chordal replacement are employed to address leaflet prolapse caused by elongated or ruptured chordae tendineae. Artificial chordal replacement has now become the most commonly used technique for degenerative/myxomatous mitral valve disease with leaflet prolapse or ruptured chordae tendineae. More recently, transcatheter valve repair devices have been developed to extend the benefits of valve repair to patients who are not candidates for open surgery, including edge-to-edge repair devices that clip the mitral leaflets together to reduce regurgitation, and transcatheter annuloplasty systems that reshape the valve annulus.

### SUMMARY

In accordance with at least some embodiments disclosed herein is the realization that the success of atrioventricular valve repair using multiple chordal sutures depends, in significant part, on the ability to manage the geometry and tension of all sutures simultaneously and reproducibly, including in a manner that is substantially independent of the operator's individual suture-tying technique.

Further, in accordance with at least some embodiments disclosed herein is the realization that significant limitations exist for conventional approaches despite the meaningful advances in atrioventricular valve repair. For example, surgical chordal replacement, while effective, requires the independent placement, tensioning, and tying of multiple individual sutures in a confined and technically demanding operative field. Because each suture is managed separately, achieving consistent and reproducible subvalvular geometry across all sutures simultaneously is difficult, and the risk of error in suture length or tension is non-trivial. Therefore, the success rate of mitral valve repair with artificial chordal replacement is extremely variable, heavily depending on the operator's experience. Annuloplasty alone does not address subvalvular pathology and may be insufficient in patients with significant chordal disease or leaflet prolapse. Furthermore, existing transcatheter repair devices, while less invasive, are generally limited in the scope of pathology they can address. And few existing devices are adaptable to the considerable variation in valve anatomy across the patient population.

The present disclosure seeks to address these limitations by reducing the complexity of multi-suture management, improving procedural reproducibility, and providing a dimensionally adaptable prosthesis that may be tailored to the anatomy of individual patients. The system can advantageously be compatible with both open surgical and transcatheter delivery approaches.

In some embodiments, a suture manifold is provided having a body, one or more inlets, one or more internal passages, and a plurality of outlets, the suture manifold configured to receive and route a plurality of sutures from the one or more inlets through the one or more internal passages and out through the plurality of outlets, such that the suture manifold organizes and directs the plurality of sutures during an atrioventricular valve repair procedure.

Additionally, in some embodiments, the suture manifold may maintain, for each of a plurality of vertical sutures, a substantially equal ratio of suture length to a respective travel displacement between an upper anchor point and a lower anchor point, such that a tautness across the plurality of vertical sutures is substantially uniform when the plurality of vertical sutures are simultaneously adjusted through an upper bundle, even where the individual lengths of the vertical sutures may differ from one another.

Further, some embodiments provide a heart valve prosthesis that comprises a suture manifold and a suture net formed by two or more vertical sutures and at least one horizontal thread, the suture net being adjustable through proximal or distal movement of an upper bundle to control a tautness of the suture net as a whole.

In accordance with some embodiments, a prosthesis delivery system is provided that comprises a delivery sheath, a prosthesis device housed within the delivery sheath, and a plurality of delivery catheters housed within the delivery sheath, each delivery catheter having a longitudinal slit through which a filament may exit the catheter laterally so as to maintain organizational integrity of filament routing within the delivery sheath.

Moreover, some embodiments relate to methods of repairing an atrioventricular valve comprises attaching a ventricle fastener to a ventricle of a heart, attaching a suture manifold to an atrium of the heart or to an annuloplasty ring, and adjusting a length of two or more sutures concurrently and substantially equally through retraction of an upper bundle, followed by concurrently locking and trimming the two or more sutures.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

While the specification concludes with claims which particularly point out and distinctly claim the inventions, it is believed the present invention will be better understood from the following description of certain examples taken in conjunction with the accompanying drawings, in which like reference numerals identify the same elements and in which:

FIG. 1 depicts a front view of an exemplary suture manifold comprising a body, an inlet, an internal passage, and a plurality of outlets, according to some embodiments.

FIG. 2 depicts a front view of another exemplary suture manifold, further comprising a second inlet, a second internal passage, and a second plurality of outlets, according to some embodiments.

FIG. 3A depicts a front cross-sectional view of another exemplary suture manifold comprising a first inlet, a second inlet, and a single internal passage, according to some embodiments.

FIG. 3B depicts a front cross-sectional view of another exemplary suture manifold comprising a first inlet, a second inlet, a first internal passage, and a second internal passage, according to some embodiments.

FIG. 3C depicts a top view of another exemplary suture manifold comprising a first inlet, a second inlet, a first internal passage, and a second internal passage, according to some embodiments.

FIG. 3D depicts a front cross-sectional view of another exemplary suture manifold comprising a first inlet, a second inlet, a third inlet, a fourth inlet, a first internal passage, a second internal passage, a third internal passage, and a fourth internal passage, according to some embodiments.

FIG. 3E depicts a front cross-sectional view of another exemplary suture manifold comprising two fluidly discontinuous internal passages, according to some embodiments.

FIG. 3F depicts a front cross-sectional view of another exemplary suture manifold comprising two fluidly discontinuous internal passages, according to some embodiments.

FIG. 3G depicts a front cross-sectional view of another exemplary suture manifold comprising two fluidly discontinuous internal passages, according to some embodiments.

FIG. 3H depicts a front cross-sectional view of another exemplary suture manifold comprising two fluidly discontinuous internal passages, according to some embodiments.

FIG. 4 depicts a front view of another exemplary suture manifold oriented along a longitudinal axis, according to some embodiments.

FIG. 5A depicts a perspective view of another exemplary suture manifold, according to some embodiments.

FIG. 5B depicts a perspective view of another exemplary suture manifold, according to some embodiments.

FIG. 5C depicts a perspective view of another exemplary suture manifold, according to some embodiments.

FIG. 6 depicts a top cross-sectional view of one or more exemplary suture manifolds when installed within the heart anatomy, according to some embodiments.

FIG. 7 depicts a front view of an exemplary suture manifold and an exemplary adjustable suture net comprising at least two vertical sutures and at least one horizontal thread, according to some embodiments.

FIG. 8 depicts a front view of another exemplary adjustable suture net comprising an upper bundle and a lower bundle, according to some embodiments.

FIG. 9 depicts a front view of an upper bundle of an exemplary adjustable suture net, according to some embodiments.

FIG. 10 depicts a perspective view of an exemplary heart valve prosthesis when installed within the heart anatomy, the prosthesis comprising a suture manifold and two or more vertical sutures, according to some embodiments.

FIG. 11A depicts a front view of a lower bundle of an exemplary adjustable suture net, according to some embodiments.

FIG. 11B depicts a front view of an exemplary suture manifold when installed within the heart anatomy, according to some embodiments.

FIG. 11C depicts a top perspective view of an exemplary suture manifold when installed within the heart anatomy, according to some embodiments.

FIG. 12A depicts a top view of an exemplary heart valve prosthesis when installed within the heart anatomy, the prosthesis comprising an annuloplasty ring and a suture manifold, according to some embodiments.

FIG. 12B depicts a front view of an exemplary heart valve prosthesis when installed within the heart anatomy, the prosthesis comprising a suture manifold, according to some embodiments.

FIG. 13 depicts a view of front view of an exemplary delivery catheter comprising a longitudinal slit, and a transport element stored within its body, according to some embodiments.

FIG. 14 depicts a cross-sectional view of two exemplary delivery catheters connected by a filament, according to some embodiments.

FIG. 15A depicts a front view of an exemplary delivery catheter with longitudinal slit having length A, according to some embodiments.

FIG. 15B depicts a cross-sectional view of an exemplary delivery catheter with a longitudinal slit having a degree of overlap X and a width of Y, according to some embodiments.

FIG. 16 depicts a front cross-sectional view of an exemplary delivery sheath comprising a prosthesis and one or more delivery catheters, according to some embodiments.

FIG. 17 depicts a front cross-sectional view of another exemplary delivery sheath comprising a prosthesis and one or more delivery catheters, according to some embodiments.

FIG. 18A depicts a cross-sectional view of an exemplary delivery sheath and the arrangement of a prosthesis and one or more delivery catheters housed within the delivery sheath, according to some embodiments.

FIG. 18B a cross-sectional view of another exemplary delivery sheath and the arrangement of a prosthesis and one or more delivery catheters housed within the delivery sheath, according to some embodiments.

FIG. 19 depicts the steps of a method of surgically repairing a heart valve, according to some embodiments.

FIG. 20 depicts the steps of a method of repairing a heart valve via a transcatheter procedure, according to some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

### Overview of the Suture Manifold

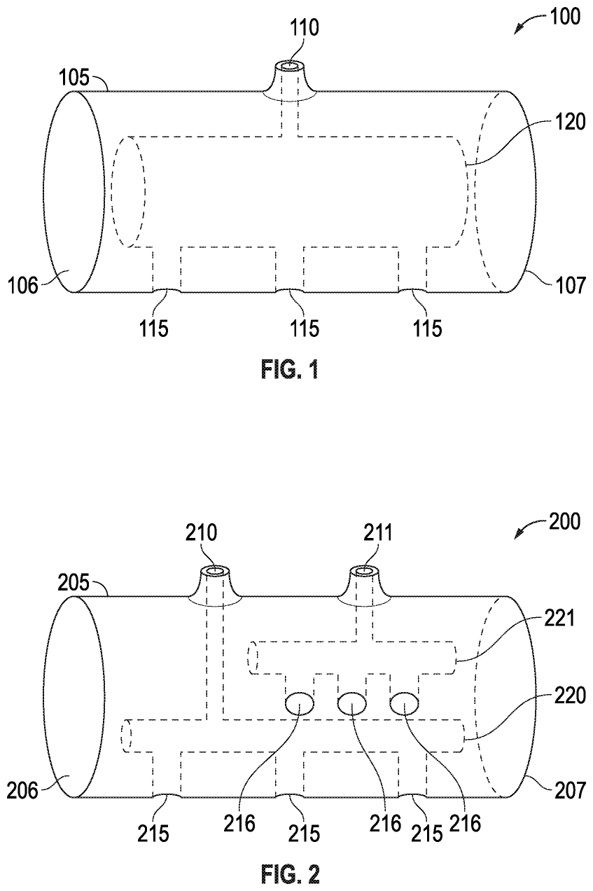

Turning to FIG. 1, there is shown a suture manifold 100 comprising a body 105, an inlet 110, an internal passage 120, and a plurality of outlets 115.

In some embodiments, the suture manifold 100 may be a medical device configured for use in mitral valve repair and/or tricuspid valve repair procedures. The suture manifold 100 may serve to direct one or more sutures through its internal passage 120 and to control the length and routing of those sutures during a repair procedure. The suture manifold 100 may include one or more inlets 110 through which sutures are received, wherein the length of each suture may be controlled at or substantially near to the inlet 110, including by lengthening, shortening, or cutting the suture as needed. Each suture travels from the inlet through one or more internal passages 120, which direct the suture through the body 105 of the manifold 100. The suture then exits the body through one of the plurality of outlets 115, the positions of which may be configured to control where and at what geometry the sutures emerge for eventual attachment to the target anatomy. In this manner, the suture manifold 100 provides a mechanism for organizing, directing, anchoring and managing multiple sutures simultaneously during a valve repair procedure.

As shown in FIG. 1, the body 105 may include a first end 106 and a second end 107. The inlet 110 may be connected to at least one of the plurality of outlets 115 through the internal passage 120. In some embodiments, the inlet 110 is connected to all of the plurality of outlets 115 through the internal passage 120. And in other embodiments, the internal passage 120 may traverse from the first end 106 through the second end 107, forming a passage completely through the body 105. In some embodiments, the suture manifold 100 may be circumferential and may circumscribe or substantially circumscribe the valve annulus (not shown). In the same or other embodiments, the suture manifold 100 may also function as a mitral annuloplasty device.

### Inlet and Internal Passage Configurations

In some configurations, the suture manifold 100 may include more than one internal passage 120. In the same or other embodiments, the two or more internal passages 120 may be “siloed,” i.e., not fluidly connected to one another. The separation of internal passages 120 may allow each passage to serve different purposes within the procedure. For example, a first internal passage may be dedicated to directing the sutures used for the valve repair itself. In some examples, a second internal passage may be associated with an anchoring mechanism for the suture manifold (i.e., to secure the suture manifold itself to a target anatomy), through which sutures for one or more anchoring elements are directed. Separating the sutures associated with the anchoring mechanism from the repair sutures may offer advantages. For example, siloing the two functions may reduce the risk of interference between the anchoring elements and the repair sutures during deployment.

In other embodiments, two or more internal passages 120 may each be dedicated to a distinct set of valve repair sutures, such that a first set of sutures travels through a first internal passage, a second set of sutures travels through a second internal passage, a third set of sutures travels through a third internal passage, and so forth, with the multiple sets of sutures remaining physically separated from one another throughout their routing within the suture manifold 100. This configuration may allow each set of sutures to be controlled, tensioned, and secured independently of the other set(s). For example, in a valve repair procedure involving a suture manifold placed near the valve commissural regions, different sets of repair sutures may be associated with the repair of different leaflets. Separating the corresponding suture sets into dedicated passages may reduce the risk of cross-interference and allow the surgeon to address each region of the repair in an independent and/or sequential manner.

Generally, the separation may allow the different sutures sets to be advanced, tensioned, and/or secured independently of one another. In some embodiments, separation of suture sets may further allow different suture types, sizes, or materials to be used within each set, and may minimize any risk of confusion.

Turning to FIG. 2, there is shown a configuration of another suture manifold 200 comprising a first inlet 210, first internal passage 220, and a first plurality of outlets 215. In addition, suture manifold 200 may comprise a second inlet 211, a second internal passage 221, and a second plurality of outlets 216. As with suture manifold 100, the first inlet 210 may be connected to at least one of or all of the first plurality of outlets 215 through the first internal passage 220. The second inlet 211 may be connected to at least one of the plurality of outlets 216 through the second internal passage 221. In some embodiments, the first inlet 210 is connected to all of the first plurality of outlets 215. In some embodiments, the second inlet 211 is connected to all of the second plurality of outlets 216. In some embodiments, the first internal passage 220 and second internal passage 221 of suture manifold 200 may each traverse from a first end 206 to a second end 207 of the body 205, each thereby forming a passage through the body 205.

### Geometry of Internal Passages

The geometry of the internal passages 220, 221, including but not limited to their cross-sectional shape, relative orientation, and spatial arrangement within the body 205 of the suture manifold 200, may be configured as necessary and by means known to those of skill in the art to accommodate the specific anchoring mechanism and/or repair suture configuration required for a given procedure. Several examples, while not exhaustive, are provided below.

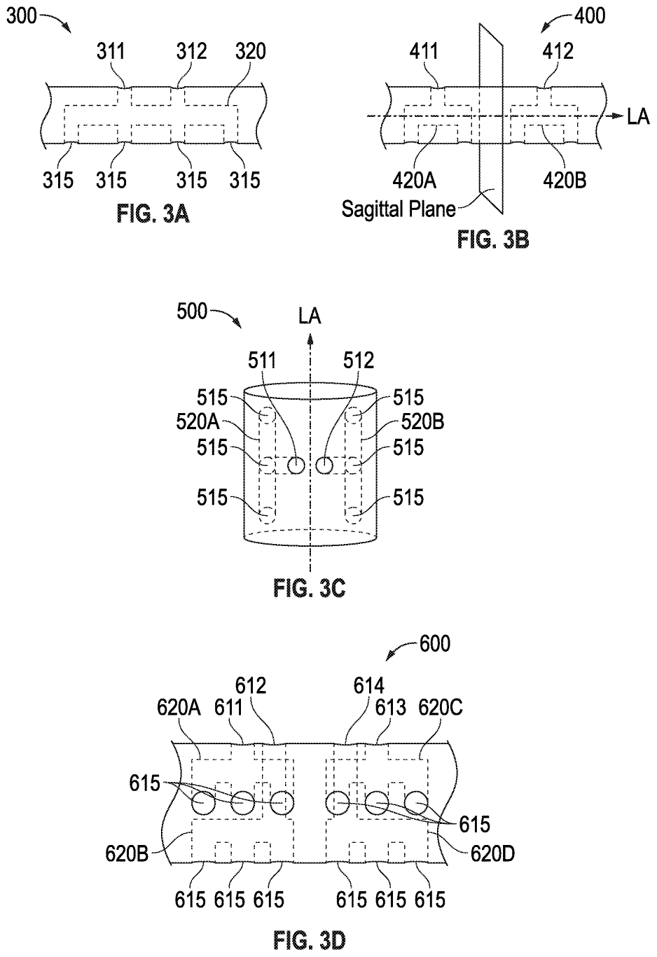

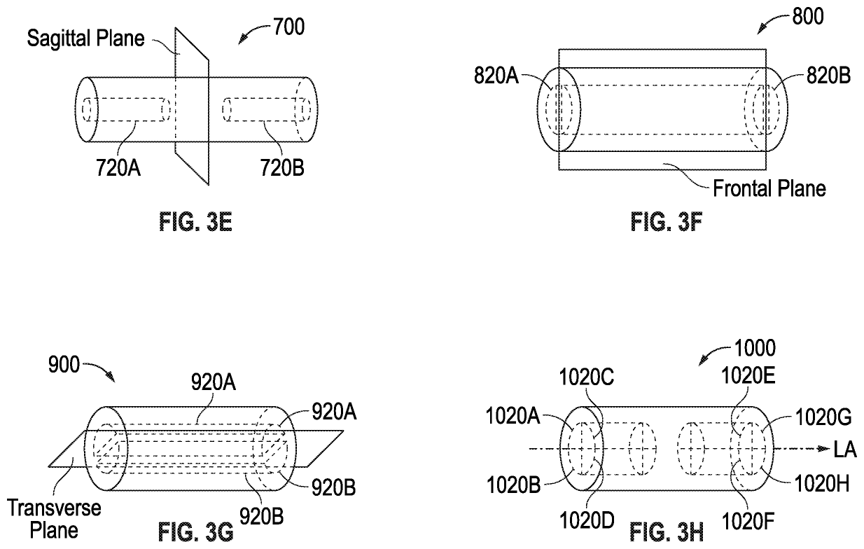

Turning to FIGS. 3A-3H, suture manifolds 300, 400, 500, 600, 700, 800, 900, and 1000 show a variety of configurations for the one or more internal passages 320, 420, 520, 620, 720, 820, 920, and 1020.

In FIG. 3A, one possible embodiment is shown in suture manifold 300, comprising a first inlet 311 and a second inlet 312, but only one internal passage 320. For example, the first inlet 311 and the second inlet 312 share one internal passage 320, which connects each inlet 311, 312 to the plurality of outlets 315.

In FIG. 3B, there is shown a configuration of suture manifold 400 in which the first internal passage 420A and the second internal passage 420B are siloed from one another. For example, the first internal passage 420A and the second internal passage 420B are not fluidly connected. Accordingly, in some embodiments, two sets of sutures may run through each of the first internal passage 420A and second internal passage 420B, respectively, and the two sets of sutures may be controlled independently.

At least some embodiments disclosed herein reflect the realization that providing two or more internal passages 120 that are physically siloed from one another within the body 105 of the suture manifold 100, with each internal passage 120 dedicated to a distinct set of valve repair sutures 155, may allow the different sets of sutures to be tensioned, secured, and otherwise managed independently of one another during the same procedure. The independent handling enabled by the siloed-passage configuration may, for example, enable a single device to deliver, in one procedure, suture sets targeting different leaflets of the same valve, different regions of a single leaflet, or different valve repair functions altogether, while avoiding entanglement or cross-interference between the sets.

The particular combination of internal passages and their geometric configuration and siloing may be determined by one of ordinary skill in the art based on the requirements of the specific procedure and the anatomy of the patient. In addition, the combination may be determined based on the number of sutures being deployed, the location at which the suture manifold is anchored to the target anatomy, the positions of the outlets through which the sutures must exit the body, and the positions of the inlets through which the sutures are received and controlled, although the above factors are not intended to be exhaustive.

In the possible embodiment shown in FIG. 3B, depicting suture manifold 400, a first internal passage 420A and the second internal passage 420B may be located sequentially along a longitudinal axis LA of the suture manifold 400.

In accordance with some embodiments, FIG. 3C depicts a suture manifold 500, a first internal passage 520A and a second internal passage 520B may be located parallel along the longitudinal axis of the suture manifold 500 but may be siloed on opposite sides of another plane of the suture manifold 500. Various planes along which the internal passages may be siloed are explored below.

Turning to FIG. 3E, there is shown an internal wall configuration of suture manifold 700 in which the first and second internal passages 720A, 720B are siloed by a sagittal plane. FIG. 3F shows an internal wall configuration of suture manifold 800 in which the first and second internal passages 820A, 820B are siloed by a frontal plane. FIG. 3G shows an internal wall configuration of suture manifold 900 in which the first and second internal passages 920A, 920B are siloed by a transverse plane. As used herein, the terms “sagittal plane,” “transverse plane,” and “frontal plane” are used in accordance with their standard definitions in medical literature. The sagittal plane is a vertical plane that divides the body or device into left and right portions. The transverse plane is a horizontal plane that divides the body or device into upper and lower portions. The frontal plane is a vertical plane that divides the body or device into anterior and posterior portions and is oriented perpendicularly to both the sagittal plane and the transverse plane. As used in the context of FIGS. 3E, 3F, and 3G of the present disclosure, the frontal plane faces the viewer of the page. These descriptions are used throughout the present disclosure for ease of understanding only and are not intended to imply any fixed absolute orientation of the device relative to the patient unless otherwise stated.

Turning to FIG. 3H, there is shown a configuration of suture manifold 1000 in which one or more of a plurality of internal passages 1020A, 1020B, 1020C, 1020D, 1020E, 1020F, 1020H, 1020G are siloed from one another by one or more radial internal walls. As used herein, the term “radial” refers to a direction extending perpendicular to the longitudinal axis LA of the body.

Returning to FIG. 3D, there is shown a configuration of the suture manifold 600 having a plurality of inlets 611, 612, 613, 614 and a plurality of internal passages 620A, 620B, 620C, 620D. Furthermore, each internal passage 620A, 620B, 620C, 620D connects one of the inlets 611, 612, 613, 614 to at least one of the plurality of outlets 615. At least one internal passage 620A, 620B, 620C, 620D of the plurality may be siloed from at least one other internal passage 620A, 620B, 620C, 620D of the plurality. In the specific embodiment shown by suture manifold 600, each of the plurality of internal passages 620A, 620B, 620C, 620D are siloed from all others. In other embodiments, however, one or more—or even all of—internal passages 620A, 620B, 620C, 620D may be fluidly connected.

While four inlets are shown in FIG. 3D, it may be appreciated that the suture manifold 600 may include any number of inlets, any number of internal passages, and any number of outlets.

In other embodiments, it may further be appreciated that a suture manifold may employ any combination of the aforementioned siloing geometries.

### Inlet and Outlet Positioning

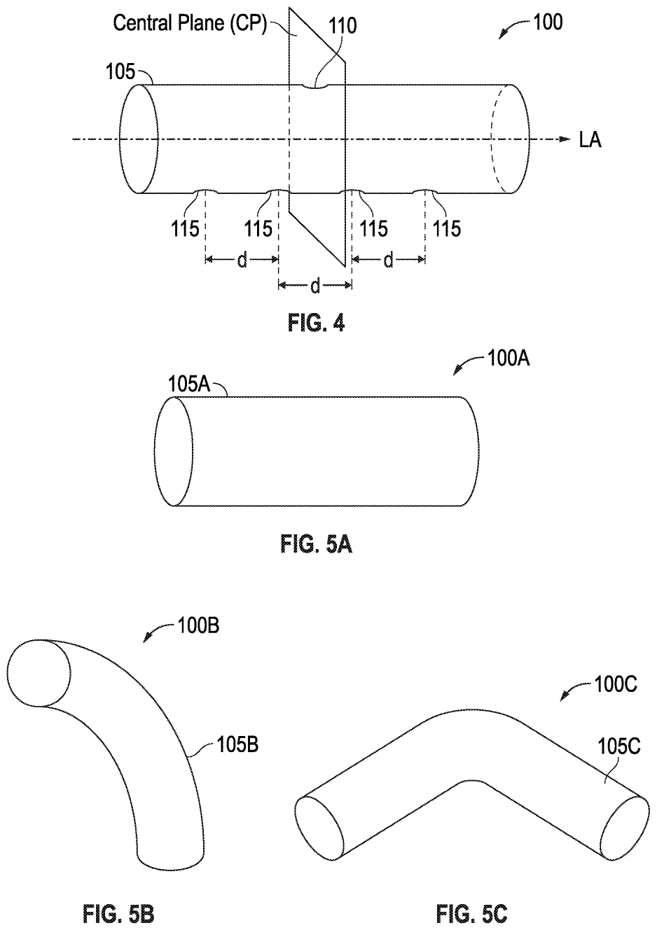

Turning to FIG. 4, there is shown an exemplary suture manifold 100 comprising the inlet 110, where the inlet 110 is located along a central plane CP of the body 105. As used herein, the term “central plane” refers to a plane oriented perpendicularly to the longitudinal axis LA of the body 105 and located at the midpoint along the axis LA. In other embodiments, however, the inlet 110 or inlets may be located off-center from the central plane CP. In some embodiments, the placement of the inlet 110 along the body 105 may be selected based on how and from what direction the operator intends to manage the length and tension of the repair sutures and/or anchoring mechanisms during the procedure. The above may also apply to a suture manifold 100 comprising a second inlet, a third inlet, a fourth inlet, and so forth.

In the embodiment shown in FIG. 4, the plurality of outlets 115 are located collinearly along the body 105 and are equally spaced from one another according to a distance d along the longitudinal axis LA of the body 105. In other embodiments, however, the plurality of outlets 115 may not be collinear and/or may be spaced unevenly with respect to the longitudinal axis LA. For example, the outlets 115 may be arranged in a helical pattern spiraling around a circumference of the cylindrical body 115. In another example, the outlets 115 may be arranged in two or more parallel rows, each row parallel to the longitudinal axis LA. In further examples, the outlets 115 may be distributed circumferentially around the body at a single longitudinal position. The above represent examples only and are not intended to limit the present disclosure. In some embodiments, the number of outlets 115, their spacing and collinearity along the body 105 may be configured to correspond to the specific anatomical locations at which the sutures are to be attached and to control the qualities of a suture net, as described in greater detail in the sections below.

In some embodiments, the geometry of the internal passages 120, and the position of the inlets 110 and outlets 115 may be selected to control a travel length and each suture's route through the manifold.

### Body Geometry

Turning to FIG. 5A, there is shown a suture manifold 100A with a body 105A having a cylindrical shape. The body 105A may be cylindrical, extending along a longitudinal axis LA with a substantially uniform cross-section. For example, a cylindrical body may be advantageous in procedures where the suture manifold is to be positioned along a relatively straight anatomical region or where a uniform profile is desirable for ease of delivery, handling and/or placement.

Turning to FIG. 5B, there is shown a suture manifold 100B with a body 105B having an arcuate cylindrical shape, where the longitudinal axis LA of the body follows a continuous, smooth curve rather than a straight line. For example, the arcuate configuration may more closely conform to the curvature of a patient's anatomy, such as along or adjacent to a portion or the entirety of the annulus of the mitral valve or the tricuspid valve. In some embodiments, the degree of curvature of the arcuate body may vary depending on the anatomy of the patient and the nature of the valve repair being performed.

In some embodiments, the suture manifold 100B may be circumferential and may circumscribe the anatomy of the entire mitral or tricuspid valve annulus. In the same or other embodiments, the suture manifold 100B may be substantially circumferential and may conform to the specific anatomical shape of the entire mitral or tricuspid valve annulus.

Turning to FIG. 5C, there is shown a suture manifold 100C having a body 105C having a bent cylindrical shape, where the longitudinal axis LA of the body includes one or more discrete angular transitions, as distinguished from the continuous smooth curvature of the arcuate cylindrical configuration. The bent configuration may be advantageous in procedures requiring the suture manifold to be oriented at a specific angle relative to the local anatomy and/or where the anatomy of the valve annulus demands a more abrupt directional change. For example, the bent cylindrical configuration may be particularly suited for use in a commissure valve repair procedure and/or where multiple valve leaflets may be targeted for repair.

More generally, the geometry of the body 105—whether cylindrical, arcuate cylindrical, bent cylindrical, circumferential cylindrical, or a different shape altogether—may be selected based on factors including the shape of the mitral or tricuspid valve annulus of the patient and the type or degree of valve repair being performed.

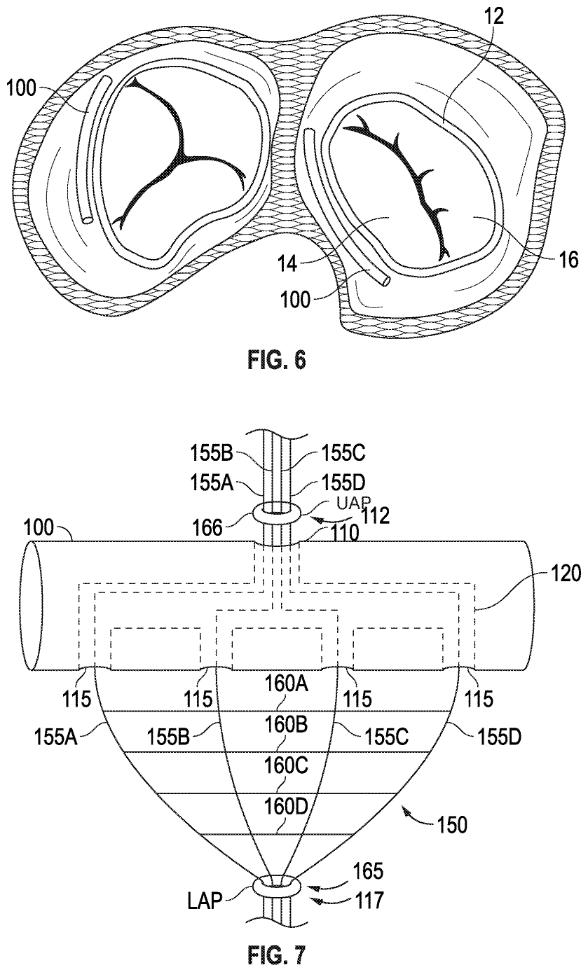

Turning to FIG. 6, the mitral valve, also known as the bicuspid valve, is a two-leaflet heart valve located between the left atrium (not shown) and the left ventricle (not shown), and includes the valve annulus 12, the anterior leaflet 14 and posterior leaflet 16, the chordae tendineae (not shown), and the papillary muscles (not shown). The valve annulus 12 forms a fibrous ring at the base of the mitral leaflets 14, 16 and may, in some embodiments, serve as the structural anchor for the suture manifold 100, although other locations are also possible and envisioned. Also seen in FIG. 6 is the tricuspid valve, a three-leaflet valve located between the right atrium (not shown) and right ventricle (not shown), which can include the chordae tendinae (not shown) and the papillary muscles (not shown). The tricuspid valve may also comprise an annulus (not shown), formed as a fibrous ring at the base of the tricuspid leaflets. In some embodiments, the tricuspid valve annulus may serve as the structural anchor for the suture manifold 100, although other locations are also possible and envisioned.

In some embodiments, the dimensions of the suture manifold 100 may be adapted to correspond to the anatomy of a particular patient, including the size and shape of the valve annulus 12 or tricuspid valve annulus, as well as the specific procedure being performed. Patient-specific adaptation of the suture manifold 100 dimensions may be informed by preoperative imaging data, which may be used to characterize the geometry of the mitral or tricuspid valve, the degree of dilation, the specific leaflet pathology, the spatial relationship between the valve and the subvalvular structures, and other factors known to those of skill in the art. Furthermore, these same factors may also contribute to determine the number of sutures to be used with the suture manifold 100 and may inform the geometric arrangement of said sutures.

For example, in some embodiments, the one or more dimensions of the suture manifold 100 may be selected to correspond to the patient's specific anatomy and intended procedure. For example, a longitudinal length of the body 105 may be between about 10 mm to about 120 mm to substantially match an arc length of a target portion of the patient's mitral or tricuspid valve annulus; a curvature of the body 105 may substantially match an annular curvature of the patient's mitral or tricuspid annulus; a number of outlets in the plurality of outlets may be between about 2 and about 20 to correspond to a number of vertical sutures 155 needed to span an abnormal leaflet area of the patient; a longitudinal spacing between adjacent outlets may be between about 1 mm to about 20 mm to substantially match a longitudinal extent of the abnormal leaflet area; and an outer diameter of the body 105 may be between about 1 mm to about 8 mm where the suture manifold 100 is configured for transcatheter delivery, or between about 1 mm to about 20 mm where the suture manifold 100 is configured for open surgical delivery.

In some embodiments, the number of sutures and/or their geometric arrangement may dictate the shape and configuration of the suture manifold 100.

In some embodiments, the mechanism by which the suture manifold 100 is introduced into the patient may be taken into account when considering the shape, materials, and design of the suture manifold 100. For example, a suture manifold 100 intended for use in open surgical repair may be designed with different (perhaps fewer) dimensional and geometric constraints than one intended for transcatheter delivery, wherein the manifold must be configured to navigate the vasculature and be deployed through a catheter system with minimal profile.

In sum, the suture manifold 100 may have one or more dimensions configured to correspond to a number of sutures passing through the body 105, and/or one or more dimensions configured to correspond to an atrioventricular valve annulus shape or a size of an abnormal leaflet area of the patient, and/or one or more dimensions configured to correspond to a specific atrioventricular valve repair procedure.

### Material and Mechanical Properties of the Suture Manifold

In one example, the body 105 of the suture manifold 100 may be semi-rigid. As used herein, “semi-rigid” refers to a body that maintains sufficient structural integrity to preserve its shape and geometry during suture placement and tying, while retaining enough flexibility to conform to the contours and/or pliability of the underlying anatomy, including the mitral valve annulus, valve leaflets, or atrial wall, without creating undue stress concentrations at the points of attachment. A semi-rigid body may resist deformation under the tension applied by multiple sutures simultaneously. In some embodiments, particularly those involving transcatheter delivery, the body 105 may be configured to be compressible for loading into a catheter delivery system and to recover its intended semi-rigid geometry upon deployment at the target site.

In some embodiments, the body 105 may be constructed from one or more biocompatible materials. For example, the body may be constructed from any biocompatible material suitable for use in cardiac surgery or transcatheter cardiac intervention. In one example, the body is constructed from expanded polytetrafluoroethylene. In another example, the body is constructed from polyethylene terephthalate (Dacron). In a further example, the body is constructed from a polyurethane. Where greater structural rigidity is required, the body may incorporate a nitinol framework. Without limitation, the materials from which the body is constructed may be selected based on factors including the intended delivery mechanism, the attachment site within the heart, the expected duration of implantation, and compatibility with sterilization processes.

In some embodiments, the dimensions of the body 105 may be adapted based on the anatomy of the patient and the specific procedure being performed. For example, the body of the suture manifold may have a longitudinal length between about 10 mm to 60 mm, or between about 20 mm to 50 mm, or between about 30 mm to 40 mm. In some embodiments, the body of the suture manifold may match a full circumference of the annulus. In such embodiments, for example, the suture manifold may have a longitudinal length between about 10 mm to about 120 mm, or between about 20 mm to about 80 mm, or between about 25 mm to about 50 mm, or between about 30 mm to about 45 mm. It should be noted that the longitudinal dimension of the suture manifold 100 need not equal the full circumference of the annulus—if the manifold 100 spans only a portion of the annulus 12, such as an anterior leaflet 14 segment depicted in FIG. 6, the relevant dimension would be a chord length or arc length of the annulus 12 rather than the full circumference. If the body 105 is arcuate or bent rather than straight, the longitudinal dimension may be defined as the arc length along the curved axis rather than a straight-line end-to-end distance. In those embodiments where the body is substantially cylindrical in shape, it may have a diameter between about 1 mm to about 20 mm, or between about 2 mm to about 12 mm, or between about 3 mm to about to 8 mm, or between about 4 mm to about 6 mm. The diameter may preferably be between about 1 mm to about 8 mm. The diameter ranges above may refer to the outer diameter of the body 105. If the body 105 is arcuate or bent rather than a straight cylinder, the diameter as defined here may apply uniformly along the curved axis. The foregoing dimensions are provided by way of example. Specific dimensions of the body may vary based on the size of the suture net, patient anatomy, procedural requirements, and clinical judgment. In some embodiments, the body 105 may be circumferential, conforming to the anatomy of the entire valve annulus. In the same or other embodiments, the body 105 may also function as an annuloplasty device. For example, the suture manifold 100 may be a part of a partial or full annuloplasty ring, as described in greater detail below.

At least some embodiments disclosed herein reflect the realization that the body 105 of the suture manifold 100 may be configured with a longitudinal length, diameter, curvature, and overall shape selected from a range of disclosed dimensions to correspond to a particular patient's valve anatomy, the particular valve being repaired (e.g., mitral versus tricuspid), the number of vertical sutures 155 being routed, and the chosen delivery approach (e.g., open surgical versus transcatheter). The disclosed dimensional adaptability of the body 105 may permit a single design platform to address the substantial variation in valve anatomy and pathology across the patient population, rather than requiring entirely separate device families for different patient subsets.

### Overview of the Suture Net

Turning to FIG. 7, there is shown an adjustable suture net 150 comprising at least two vertical sutures 155 (four shown, 155A, 155B, 155C, 155D), each vertical suture 155 being directly or indirectly fixed at an upper anchor point UAP (e.g., a relative position of the suture manifold where it is affixed to the tissue or a position on the suture manifold, such as where the sutures are bundled, or any suitable position relative thereto) and at a lower anchor point LAP (e.g., a relative position at which the papillary anchor is affixed to the tissue or a position above that point where the sutures are bundled together, or any suitable position relative thereto). In some embodiments, at least one horizontal thread 160 (four shown, 160A, 160B, 160C, 160D) may tether together one or more vertical sutures 155. The vertical sutures 155 may pass through the inlet 110, internal passage(s) 120 of the suture manifold 100, and outlets (115) of the suture manifold 100.

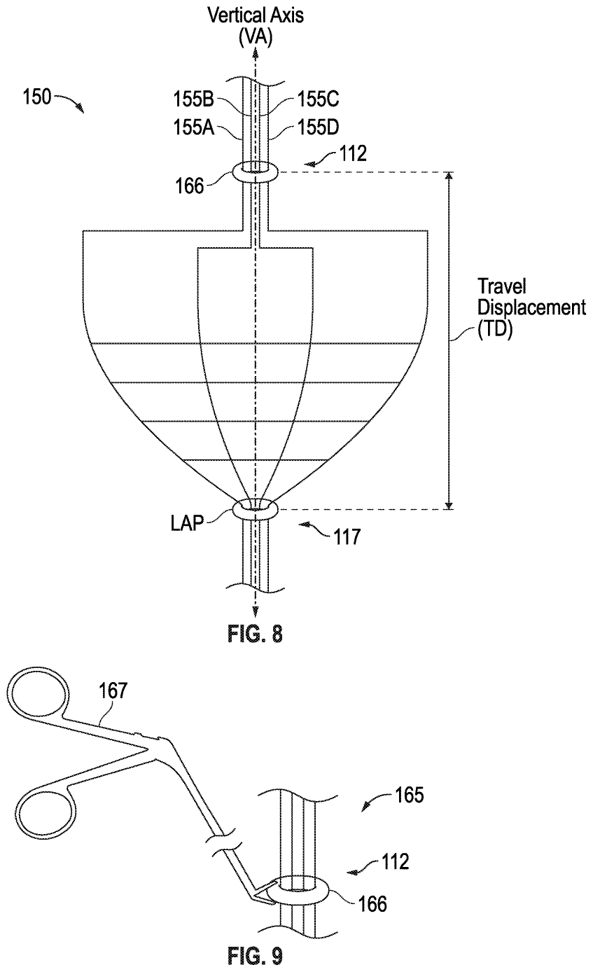

With reference to FIGS. 7 and 8, the upper anchor point UAP and the lower anchor point LAP may establish the upper and lower boundaries, respectively, between which each vertical suture 155 travels to at least partially establish the suture net 150. The upper anchor point UAP may be configured to be fixed relative to the suture manifold 100, for example at or near the inlet 110, and the suture manifold itself may be configured to be fixed to local anatomy, including but not limited to the mitral or tricuspid valve annulus, the valve leaflets, or the atrial tissue. Accordingly, the UAP may be fixed relative to the local anatomy of the atrium. The lower anchor point LAP may be fixed relative to local anatomy at or near the ventricular side of the valve, including but not limited to the papillary muscle, the ventricular muscle or ventricular tissue. A travel displacement TD between the upper anchor point UAP and the lower anchor point LAP may be established and remain fixed after complete deployment and final adjustment of the suture net 150.

At least some embodiments disclosed herein reflect the realization that the travel displacement TD between the upper anchor point UAP and the lower anchor point LAP may be decoupled from the manipulation of the upper bundle 112 by the suture manifold 100. In conventional chordal replacement techniques, adjusting the length or tension of any individual suture requires direct manipulation of the suture at or near the valve anatomy, which is in close proximity to the travel displacement TD. By contrast, in at least some embodiments disclosed herein, the upper bundle 112 may be manipulated at a location proximal to and spatially separated from the upper anchor point UAP, such that the suture lengths within the suture net 150 may be adjusted without disturbing the anchoring points and without requiring direct surgical access to the valve anatomy.

Notwithstanding the fixed nature of the travel displacement TD after complete deployment and final adjustment, the length of a given suture 155 traveling between the upper anchor point UAP and the lower anchor point LAP may vary independently of the travel displacement TD. Specifically, a vertical suture 155 spanning the travel displacement TD may be rendered taut, wherein the suture length decreases, or may be rendered slack, wherein the suture length increases. For example, excess suture length may manifest as slack within the net. The adjustability of each vertical suture's length therefore allows the tautness of the suture net 150 to be precisely controlled after both anchor points UAP, LAP have been secured, without altering the positions of the anchor points UAP, LAP. The decoupling of travel displacement TD from suture length may better enable the doctor to fine tune suture tension after initial placement of the suture manifold 100.

### Length of the Vertical Sutures

In some embodiments, a length of each vertical suture 155 between the upper anchor point UAP and the lower anchor point LAP is adjustable. In some embodiments, the length of each suture 155 controls the tautness of the same suture 155. In some embodiments, the length of each suture 155 is proportional to the tautness and/or tension of the same suture 155.

In some embodiments, the length of each vertical suture 155 controls an overall tautness of the entire suture net 150. In some embodiments, the length of each suture 155 is proportional to the overall tautness of the entire suture net 150.

As used herein, the terms “vertical” and “horizontal” are defined relative to the suture manifold 100, which is oriented substantially horizontally when deployed at the target anatomy. Accordingly, the horizontal threads 160 of the adjustable suture net 150 may extend in a direction substantially parallel to the longitudinal axis LA of the suture manifold 100. The vertical sutures 155 may extend in a direction substantially perpendicular to the longitudinal axis LA of the suture manifold 100. Specifically, the vertical sutures 155 may span the travel displacement TD between the upper anchor point UAP and the lower anchor point LAP, where the travel displacement TD is oriented substantially vertically. It will be understood that “vertical” and “horizontal” as used herein are relative terms of orientation with respect to the suture manifold 100 after installation and are not intended to imply any absolute spatial orientation with respect to the patient or the operation.

In some embodiments, the horizontal threads 160 may connect and tether one or more, or even all of, the vertical sutures 155 to one another, thereby forming the structure of the adjustable suture net 150. The manner by which the horizontal threads 160 are connected to the vertical sutures 155 may be accomplished by any means known to those of ordinary skill in the art, including but not limited to nets, ties, or knots, and the present disclosure is not intended to be limited to any particular connection method.

In certain embodiments, the tautness or slackness of the horizontal threads 160 can be determined by the tautness or slackness of the vertical sutures 155. In such embodiments, as the length of each vertical suture 155 is adjusted—for example, by lengthening or shortening—the resulting change in vertical suture 155 tautness propagates through the connection points between the vertical sutures 155 and the horizontal threads 160, proportionally adjusting the tautness of the horizontal threads 160. In this manner, the geometry and tension of the suture net 150 as a whole, including both its vertical and horizontal components, may be controlled through adjustment of the vertical suture lengths 155 alone. In other embodiments, the tautness of the vertical sutures 155 and the tautness of the horizontal threads 160 may be independent of one another.

As discussed above, the “length” or “travel length” of each vertical suture 155 can refer to the total distance traveled by the suture between the upper anchor point UAP and the lower anchor point LAP, as distinguished from the travel displacement TD, which represents the fixed linear distance between the upper anchor point UAP and the lower anchor point LAP as secured in situ. Because the vertical suture 155 may follow a non-linear path through the internal passage 120 of the suture manifold 100 and/or may travel along a hypotenuse of the substantially triangular shape of the suture net 150, the length of the suture 155 may exceed the travel displacement TD even when the suture 155 is fully taut. Notwithstanding the fact that individual suture lengths may differ from the travel displacement TD, in some embodiments the suture manifold 100 is configured so that all of the vertical sutures 155 are adjusted together—concurrently and by the same incremental distance—through proximal or distal movement of an upper bundle of the suture net, as described in greater detail below.

In some embodiments, a total length of each vertical suture 155 between the upper anchor point UAP and the lower anchor point LAP can differ based on the position of the UAP and the LAP relative to the suture manifold 100. Thus, the lengths can vary across all vertical sutures 155 of the adjustable suture net 150 or be identical (as can be generally seen in the embodiment of FIG. 7, which shows a mirror-image in which the lengths of sutures 155A and 155D are identical and the lengths of sutures 155B and 155C are identical and different from the lengths of sutures 155A and 155D. However, even where the total length of the sutures may differ, the travel length of the sutures can be substantially equal.

The total length of a given suture can be configured by designing the geometry of the internal passage(s) 120 of the suture manifold 100 and/or providing specific locations of the UAP and/or LAP. For example, the design can adjust the path of travel for each individual vertical suture 155 through the internal passage(s) 120 to offset for differences in length created by exiting at an outlet 115 to be closer to or further away from the central plane CP of the body 105 or through different internal passages 120 and exiting at outlets 115 of different internal passages 120. In the same or other embodiments, the length of each vertical suture 155 can be proportional to the travel displacement TD.

In some embodiments, the tautness of each vertical suture 155 can advantageously be maintained substantially constant across all vertical sutures 155 as the vertical sutures 155 are simultaneously lengthened or shortened, even where the respective lengths of the individual vertical sutures 155 may be unequal.

In some embodiments, a minimum travel length may be defined for each vertical suture 155 as the length of that suture 155 when it is in a maximally taut configuration, i.e., where the suture spans the travel displacement TD with substantially no slack. In some embodiments, as the sutures 155 are lengthened or shortened, the length of each vertical suture 155 may remain proportional relative to its own minimum travel length, such that the ratio of current suture length to its minimum travel length can be substantially equal across all vertical sutures 155. In the same or other embodiments, a tension and/or a tautness of the vertical suture 155 is proportional to this ratio. In this manner, the tautness of each vertical suture 155 may be kept substantially equal during adjustment, and the suture net 150 as a whole may be uniformly tensioned or relaxed through a single lengthening or shortening of all vertical sutures 155, regardless of variations in length among the individual vertical sutures 155. Thus, as discussed herein, some embodiments can be configured to have the suture length be directly or inversely proportional to the tautness and/or be directly proportional to the travel displacement. Thus, the longer the suture, the lower the tautness, or the longer the suture, the greater the tautness, as desired by the clinician. Further, the longer the suture, the greater the travel displacement or travel length or the shorter the suture, the lower the travel displacement, as desired by the clinician.

### Bundling the Vertical Sutures at the Upper Bundle

Next, with continued reference to FIGS. 7 and 8, the adjustable suture net 150 may further comprise an upper bundle 112 tethering together an upper end of each vertical suture 155, and a lower bundle 117 tethering together a lower end of each vertical suture 155.

The upper bundle 112 may be formed by bundling together the upper ends of all vertical sutures 155 at a point equal or distal to the upper anchor point UAP, such that the upper anchor point UAP serves as a common fixed point of reference for all of the vertical sutures 155. By consolidating the vertical sutures into a single upper bundle 112, all of the sutures 155 may be proximally withdrawn or distally advanced together as a unified structure, thereby facilitating uniform and simultaneous control over the length and/or tautness of all vertical sutures 155. This unified actuation may ensure that adjustments made to the upper bundle 112, i.e., advancement or retraction of the upper bundle 112, propagate consistently across each vertical suture 155 of the net 150, allowing the tautness of the suture net 150 as a whole to be controlled through a single proximal or distal movement. Further advantageously, this unified actuation can overcome the appearance, suggested by individual-path geometry, that each suture must be independently managed.

As used herein, the terms “distal” and “proximal” are defined relative to the anatomy of the heart in the context of the implanted device and, more specifically, in the context of a transcatheter procedure. In a transcatheter approach, a prosthesis and its delivery system may be advanced through the vasculature, for example through the femoral vein, jugular vein, femoral artery and the aortic arch. In such an approach, the direction of advancement may be referred to as “distal,” i.e., toward a direction or location closer to the ventricle of the heart. The direction of retraction may be referred to as “proximal,” i.e., toward a direction or location closer to the atrium of the heart.

### Bundling the Vertical Sutures at the Lower Bundle

Similarly, the lower bundle 117 may be formed by bundling together the lower ends of all vertical sutures 155 at a point equal or proximal to the lower anchor point LAP, such that the lower anchor point LAP serves as a common fixed point of reference for all of the vertical sutures 155 at the distal end of the suture net 150. By consolidating the vertical sutures into a single lower bundle 117, all vertical sutures 155 may be unified at a common distal terminus, allowing them to be collectively anchored to a single point in the local anatomy. In some embodiments, the lower bundle 117 unifies the distal ends of the vertical sutures 155 such that the anchoring force applied at the lower anchor point LAP is distributed evenly across all vertical sutures 155 of the net 150.

In some embodiments, once the lower bundle 117 is anchored at the lower anchor point LAP, the length and/or tautness of each vertical suture 155 may be adjusted by distally advancing or proximally withdrawing the upper bundle 112.

Turning now to FIG. 9, the adjustable suture net 155 may further comprise a suture management device 165. In some embodiments, the suture management device 165 may further include a fastening device 166 and/or a cutting device 167. In some embodiments, the suture management device 165 may be configured to adjust the length of each vertical suture. In some embodiments, the suture management device 165 may be configured to lock the length of each vertical suture 155, which may for example be accomplished via the fastening device 166. In some embodiments, the suture management device 165 may be configured to remove any excess suture length above the fastening device 166, which may for example be accomplished via the cutting device 167.

In some embodiments, the fastening device 166 is configured to tie a knot around the bundled upper ends of the vertical sutures 155 at the upper bundle 112, thereby permanently fixing the length of each vertical suture 155 and/or locking the tautness of the suture net. In some embodiments, once the knot is tied, the travel length of each vertical suture 155 is rendered permanent, and the suture net 150 is secured in its final operative geometry. The knot may be accomplished by any means known to those of ordinary skill in the art of surgical suture management. Examples of suitable knotting techniques include, but are not limited to, the square knot, the surgeon's knot, the sliding knot, and the Aberdeen knot. In other embodiments, the fastening mechanism 166 may employ a mechanical locking element, such as a suture clip, a cinching collar, or a pledget-based locking construct, to achieve permanent fixation of the bundled sutures 155. In minimally invasive or transcatheter applications, the locking mechanism 166 may be incorporated into a dedicated locking catheter configured to deliver and actuate the locking element 166 at the upper bundle 112 through a single dedicated delivery catheter or sheath, which will be discussed in greater detail below.

In some embodiments, the cutting device 167 is configured to sever any excess suture length remaining proximal to the fastening device 166, thereby eliminating suture tails that could otherwise interfere with surrounding cardiac structures or complicate the operation. The cutting device 167 may utilize any number of means known to those of ordinary skill in the art of surgical suture management. Examples of suitable cutting mechanisms include, but are not limited to, suture scissors, a suture cut wire, a thermal cutting element, a laser cutting element, or a blade-based suture cutter integrated into a delivery instrument. In minimally invasive or transcatheter applications, the cutting mechanism 167 may be incorporated into a dedicated suture cutting catheter configured to deliver and actuate the cutting mechanism 167 at the upper bundle 112 through a single dedicated delivery catheter or sheath, which will be discussed in greater detail below.

In certain embodiments, the fastening device 166 and cutting device 167 may be combined into a single integrated device within the suture management device 165 that is configured to tie and cut the sutures in a single sequential operation. In minimally invasive or transcatheter applications, the suture management device 165 may be incorporated into a dedicated suture management catheter configured to deliver and sequentially actuate both the locking mechanism 166 and the cutting mechanism 167 at the upper bundle 112 through a single dedicated delivery catheter or sheath, which will be discussed in greater detail below.

### Overview of the Heart Valve Prosthesis

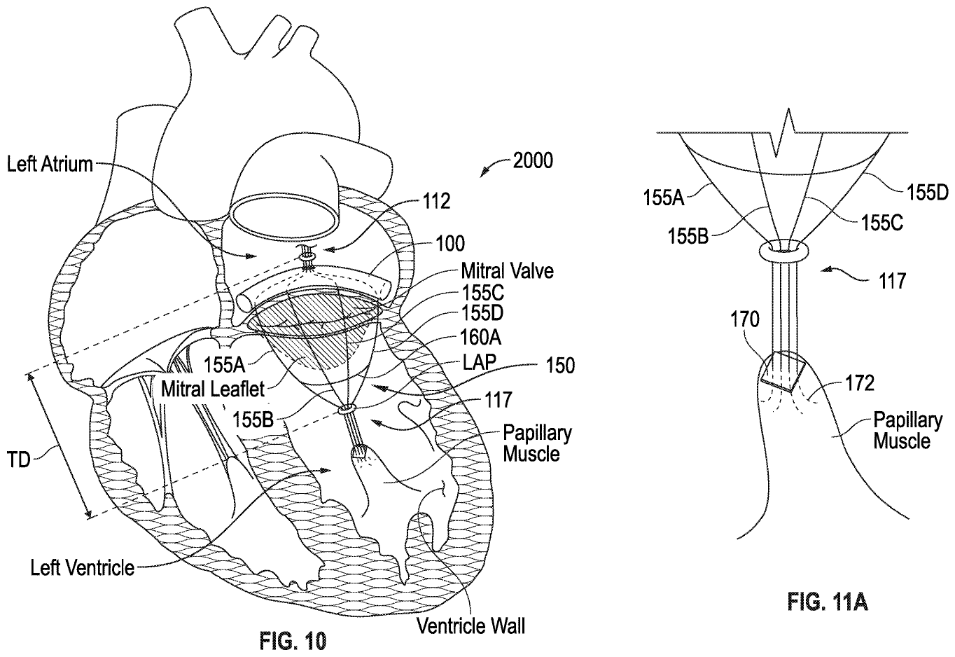

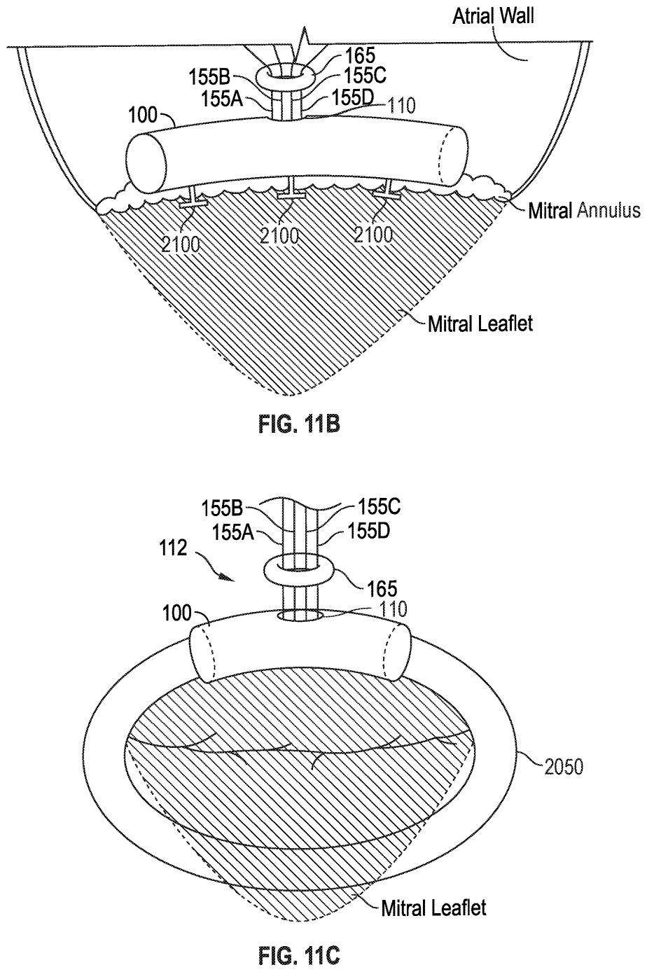

Turning to FIG. 10, there is shown a heart valve prosthesis 2000 comprising two or more vertical sutures 155 (four shown here, 155A, 155B, 155C, 155D), each suture 155 having an upper end configured to be directly or indirectly fastened to an atrium of the heart and a lower end configured to be directly or indirectly fastened to the ventricle of the heart, and a suture manifold 100 directing a path of each of the two or more sutures 155 and controlling a travel length of each suture 155. In the embodiment shown, the heart valve prosthesis 2000 may be installed on the mitral valve located in the left atrium, and the lower end of each suture 155 may be fastened to the left ventricle. For example, the lower end may be fastened to the papillary muscle. In an alternative example, the lower end may be fastened to the ventricle wall. The heart valve prosthesis 2000 may include a suture net 150, comprising the sutures 155, and supporting the mitral leaflets of the mitral valve. In some embodiments, the lower end of each suture 155 may be bundled into a lower bundle 117 distal to the suture net 150, and the upper end of each suture 155 may be bundled into an upper bundle 112 proximal to the suture manifold 100. While the heart valve prosthesis 2000 is applied to the mitral valve in FIG. 10, it may be appreciated that the heart valve prosthesis 2000 may additionally or alternatively be applied to the tricuspid valve (not shown), whereby each suture 155 may be fastened at an upper end to the right atrium (not shown) and fastened at a lower end to the right ventricle (not shown).

The heart valve prosthesis 2000 may be a surgical or transcatheter implant configured for use in the repair of atrioventricular valves. The prosthesis 2000 may replicate or augment the function of the native valve and subvalvular support structure by providing a network of vertical and horizontal components which form a net and which span from the proximal anatomy at or near the valve annulus to the distal ventricular anatomy.

The prosthesis 2000 may comprise two or more vertical sutures 155, each spanning a travel displacement TD between an upper anchor point UAP and a lower anchor point LAP. The upper end of each vertical suture 155 may be configured to be directly or indirectly fastened to the atrial anatomy, such as but not limited to the mitral or tricuspid valve annulus, the valve leaflets, or the atrial wall. The lower end of each suture 155 may be configured to be directly or indirectly fastened to the ventricular anatomy, such as but not limited to the papillary muscle or the ventricle wall.

The suture manifold 100 may direct the path of each vertical suture 155 through its internal components, routing each suture from an inlet 110, through one or more internal passages 120, and out through a dedicated outlet of a plurality of outlets 115.

At least some embodiments disclosed herein reflect the realization that, by routing the plurality of vertical sutures 155 through the suture manifold 100 and consolidating their upper ends into a common upper bundle 112, the surgeon's task of adjusting and tensioning multiple sutures may be reduced to the adjustment of a single upper bundle 112. This reduction may yield reproducible suture geometry across operators of varying surgical experience, in contrast to conventional chordal replacement techniques in which the placement, tensioning, and tying of each individual suture is performed independently and is therefore heavily dependent on the operator's individual technique. The reproducibility provided by at least some of the embodiments disclosed herein may also reduce the time required to perform the procedure and the variability of clinical outcomes.

The vertical sutures 155 may be interconnected by one or more horizontal threads 160 to form a net structure 150 below the suture manifold 100, and spanning the space between the valve annulus and the ventricular anchoring points. The tautness of this suture net 150 may be adjusted by proximally withdrawing or distally releasing an upper bundle 112 before a length of each suture—and proportionately the tautness of the suture net 150—is permanently fixed by a suture management device 165.

### Lower Bundle and Ventricular Fastening

Turning to FIG. 11A, there is depicted a lower end of each vertical suture 155 bundled into a lower bundle 117 before being fastened to the ventricular anatomy of the heart. Bundling the vertical sutures 155 may consolidate the individual sutures 155 into a unified structure, i.e., lower bundle 117, allowing all vertical sutures 155 to share a common anchoring point at the LAP. In some embodiments, the bundling may also ensure that the anchoring force applied at the ventricular anatomy is distributed uniformly across all of the vertical sutures 155.

With continued reference to FIG. 11A, the lower bundle 117 may be fastened directly or indirectly to a papillary muscle or a ventricular wall. The lower bundle 117 may be formed by various means known to those of ordinary skill in the art of surgical suture management. Examples of suitable bundling techniques include, but are not limited to, tying the lower ends of the sutures 155 together using a surgical knot; wrapping the lower ends together with a separate suture or ligature; threading the lower ends through a common pledget, eyelet, or ferrule; crimping the lower ends together using a suture crimp or metallic sleeve; or coupling the lower ends to a common anchor element such as a suture plate, a barbed anchor, nitinol tissue anchor, or a helical coil anchor, where the common anchor element is itself fixed to the ventricular anatomy.

In one exemplary embodiment, the lower bundle 117 may be fastened to a pledget 170, or similar reinforcing element, through which each suture 155 passes. The pledget may be configured as a flat pad, a folded strip, or a rolled bolster, and the like, and may be secured to the ventricular anatomy by passing the lower ends of the vertical sutures 155 through the pledget 170 and the ventricular anatomy, and tying them on its far surface. In the same or other embodiments, the pledget 170 or reinforcing element may distribute the tension of the sutures 155 over a broader area of tissue. Advantageously, the pledget 170 may reduce the risk of suture pull-through or tissue tearing at the anchoring point.

In another exemplary embodiment shown in FIG. 11A, the lower bundle 117 may additionally or alternatively be fastened to a ventricular anchor 172 that is fixed to the ventricular anatomy. The ventricular anchor 172 may take any of a variety of forms known in the art, including but not limited to a helical coil anchor, a barbed anchor, a toggle bolt anchor, or the like. The ventricular anchor 172 may be constructed from nitinol, stainless steel, cobalt-chromium alloy, or other biocompatible materials suitable for permanent or long-term implantation in the cardiac environment. The ventricular anchor 172 may be particularly advantageous in transcatheter delivery applications, where direct surgical access to the ventricular anatomy is not available and the anchor must be deployed and secured through a catheter system.

### Adjustable Suture Net Structure

With reference to FIG. 10, the heart valve prosthesis 2000 further includes a suture net 150 located below the suture manifold 100 and formed by two or more vertical sutures 155 and at least one horizontal thread 160 connecting one or more vertical sutures 155.

As discussed in detail above, the tautness of the suture net 150 may be controlled by the length of each vertical suture 155.

As used herein, the term “net density” may characterize the structural and mechanical properties of the suture net 150, determined collectively by the number of vertical sutures 155, the number of horizontal threads 160, and the tautness of the net 150. The number of vertical sutures 155 determines the horizontal resolution of the net 150 while the number of horizontal threads 160 determines the vertical resolution of the net 150. Together, these parameters may establish the overall density, rigidity, and spatial coverage of the net structure 150. A higher net density may provide more uniform leaflet support and greater resistance to prolapse, while a lower net density may provide a more compliant and anatomically conforming support structure. The net density may be adjusted according to the specific atrioventricular valve repair procedure being performed, including, for example, the nature and degree of leaflet pathology, the size and/or shape of the diseased leaflet, the location of the diseased leaflet, the size and/or shape valve annulus and/or leaflets, the number, size and geometry of sutures required, and among other possible considerations.

### Suture Length Management Through the Suture Manifold

As discussed above, the suture manifold 100 may control the path and length of each vertical suture 155 as it travels through an internal passage 120 of the suture manifold 100. The reader is referred to the sections above for a full discussion of how the suture manifold directs each vertical suture 155.

In accordance with some embodiments, a “length” or “travel length” of each suture can refer to the total distance traveled by the suture between the upper anchor point UAP and the lower anchor point LAP, which may exceed the travel displacement TD where the suture follows a non-linear path through the internal passage 120 of the suture manifold 100.

In one embodiment, the length of each suture 155 is maintained to be equal by the suture manifold 100. In one embodiment, the length of each suture 155 is maintained to be proportional to the minimum travel length of each suture 155 by the suture manifold 100. For example, a ratio of the length of each suture 155 to its minimum travel length may be maintained to be equal by the suture manifold 100. In one embodiment, a tautness and/or tension of each suture 155 is maintained to be equal by the suture manifold 100.

Thus, in some embodiments, the suture length may be directly or inversely proportional to the tautness; thus, the longer the suture, the lower the tautness, or the longer the suture, the greater the tautness, as desired by the clinician. Additionally, in some embodiments, the suture length may be directly proportional to the travel displacement; thus, the longer the suture, the greater the travel displacement or travel length or the shorter the suture, the lower the travel displacement, as desired by the clinician.

At least some embodiments disclosed herein reflect the realization that a substantially uniform tautness may be achieved across the plurality of vertical sutures 155 even where the individual lengths of the vertical sutures 155 differ from one another, provided that the ratio of each suture's length to its respective travel displacement TD is maintained substantially equal across the plurality of vertical sutures 155. Because patient anatomy may vary across leaflets and across patients, the travel displacement TD for each vertical suture 155 may differ from one suture to another in a given procedure. Maintaining equal length-to-travel-displacement ratios via the suture manifold 100, rather than attempting to set equal absolute lengths, may therefore accommodate the natural variation in subvalvular anatomy while still producing a uniform tautness suitable for valve repair.

In some embodiments, the vertical sutures 155 may be divided into one or more subsets. Accordingly, all of the characteristics described above with reference to the shared qualities of the vertical sutures 155 as a whole, e.g. their equivocal or proportional length, tautness, and the like, may apply instead to vertical sutures 155 within a single subset, where such shared qualities may not apply between vertical sutures 155 across different subsets. Thus, in some embodiments, the length of vertical sutures 155 within each subset may be maintained to be equal by the suture manifold 100. In one embodiment, a ratio of the length of each suture 155 to its minimum travel length may be maintained to be equal within each subset by the suture manifold 100. In one embodiment, a tautness and/or tension of each suture 155 within each subset is maintained to be equal by the suture manifold 100.

The geometry of one or more internal passages 120 of the suture manifold 100 may be configured to enforce these equivocal or proportional relationships.

### Suture Manifold Inlet, Internal Passage, and Outlets

The inlet, internal passage, outlet, multi-inlet, multi-passage, and siloed passage configurations of the suture manifold as described in detail in the section directed to the suture manifold 100 above likewise incorporated into the heart valve prosthesis 2000. Accordingly, the reader is referred to the sections for a full discussion of how each suture 155 may be routed from an inlet 110 through one or more internal passages 120 to a dedicated outlet 115.

In some embodiments, the suture manifold 100 of the heart valve prosthesis 2000 includes an inlet 110, an internal passage 120, and a plurality of outlets 115. In the same or other embodiments, at least one vertical suture 155 passes through the inlet 110, the internal passage 120, and one of the plurality of outlets 115. In the same or other embodiments, no two sutures 155 pass through the same outlet 115. In the same or other embodiments, a number of outlets 115 is equal to a number of vertical sutures 155, such that there is a one-to-one correspondence between the outlets 115 and the vertical sutures 155.

In some embodiments, the suture manifold 100 may comprise more than one internal passage 120. In the same or other embodiments, at least one of the internal passages 120 may be siloed from another of the internal passages 120.

In some embodiments, such as those in which the vertical sutures 155 may be divided into subsets, the suture manifold 100 may include at least two internal passages 120, wherein the sutures 155 of each subset pass through a different and/or dedicated internal passage 120. In the same or other embodiments, each subset of the vertical sutures 155 may pass through a different and/or dedicated inlet 110.

### Upper Bundle and Atrial Fastening

The structure, formation, and function of the upper bundle 112 as incorporated into the heart valve prosthesis 2000 are substantially the same as described in the section directed to the adjustable suture net 150 above, and the reader is referred to that section for a full discussion of how the upper ends of the vertical sutures 155 are consolidated into an upper bundle 112 and how proximal withdrawal or distal release of the upper bundle 112 may, in some embodiments, uniformly adjust the tautness of the suture net 150.

In some embodiments, an upper end of each suture 155 is bundled into an upper bundle 112 at a point proximal to the inlet 110 of the suture manifold 100.

### Annuloplasty Ring

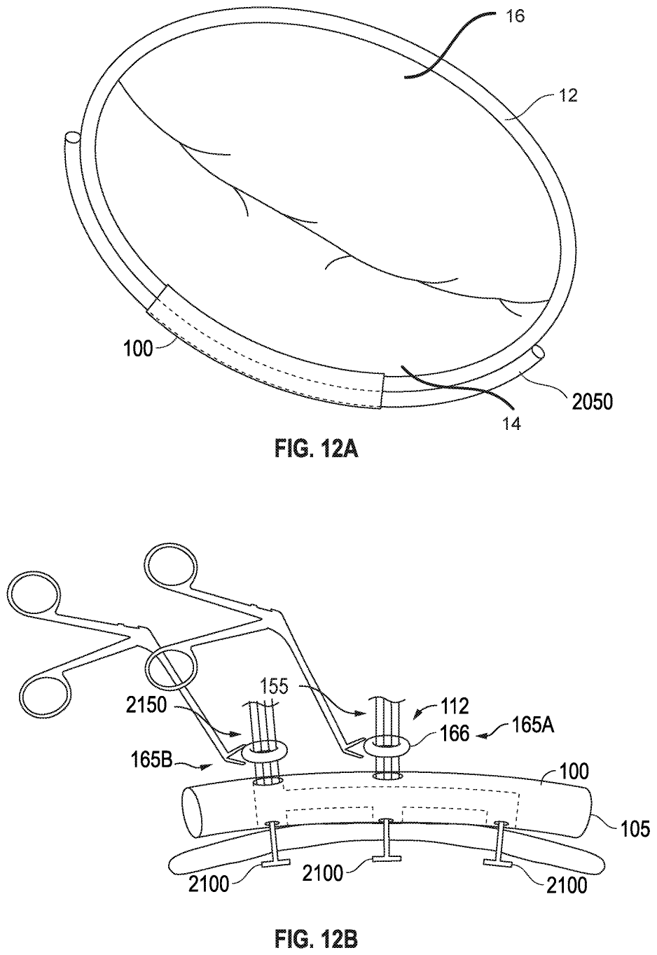

With reference now to FIGS. 11B, 11C, and 12A in some embodiments, the upper end of each vertical suture 155 is configured to be fastened to the proximal anatomy at or near the atrioventricular valve repair site, which may include the mitral annulus, the mitral leaflet, or the atrial wall, or their anatomical analogues in the tricuspid valve. In some embodiments, the selection of a fastening target may be informed by preoperative imaging, intraoperative assessment of tissue quality, and the specific requirements of the valve repair procedure, among other possible factors.

FIG. 11B depicts a front view of an exemplary suture manifold 100 that is fastened directly to the mitral annulus. FIG. 11C depicts a top perspective view of an exemplary suture manifold 100 that is fastened to an annuloplasty ring 2050. In both examples, the suture manifold 100 may sit in the left atrium of the heart above the mitral leaflet(s) which open and close to allow blood flow from the left atrium to the left ventricle. In both examples, the suture manifold 100 may comprise an inlet 110 through which one or more vertical sutures 155 pass. In some embodiments, the vertical sutures 155 may pass through a suture management device 165 before passing through the inlet 110. In alternative embodiments, the suture manifold 100 may be installed to repair the tricuspid valve (not shown), and the suture manifold 100 and/or annuloplasty ring 2050 may be affixed to analogous anatomy in the right atrium (not shown).

Turning now to FIG. 12A, in some embodiments, the heart valve prosthesis 2000 further comprises an annuloplasty ring 2050. In some embodiments, the suture manifold 100 may be directly fastened to the annuloplasty ring 2050 and the annuloplasty ring 2050 may in turn be fastened to the mitral annulus or other atrial tissue. The annuloplasty ring 2050 may be a prosthetic ring or band configured to be implanted at or near the valve annulus. By fastening the suture manifold 100 to the annuloplasty ring 2050 rather than directly to the native tissue, the prosthesis 2000 may benefit from additional structural stability, load-distributing properties, and ease of operation. In some embodiments, the annuloplasty ring 2050 contains the suture manifold 100, which provides combined valve repair techniques-annuloplasty repair with the annuloplasty ring 2050 and chordal repair with the heart valve prosthesis 2000. In some embodiments, the annuloplasty ring 2050 and suture manifold 100 may be delivered through a catheter system without requiring open surgery.

In some embodiments, such as the one shown in FIG. 11C, the annuloplasty ring 2050 may be a complete ring encircling the full circumference of the valve annulus. In other embodiments, such as the one shown in FIG. 12A, the ring 2050 may be an incomplete ring or partial band spanning only a portion of the annulus. In some embodiments, the suture manifold 100 may be part of the complete or partial annuloplasty ring 2050. In various embodiments, the ring 2050 may be semi-rigid or flexible in construction. The annuloplasty ring 2050 may be preferred in surgical operations, as direct visualization and access to the valve annulus may allow the ring 2050 to be precisely positioned. With a surgical approach, the annular ring could be the annuloplasty ring 2050 or various types of regular surgical rings.

### Suture Manifold Anchors

As shown in FIGS. 11B and 12B, in some embodiments, the suture manifold 100 may be fastened directly to the mitral annulus, the mitral leaflet, or the atrial wall via one or more manifold anchors 2100, without the intermediary of an annuloplasty ring 2050. The manifold anchors 2100 may provide a direct and dedicated fixation construct for securing the suture manifold 100 to the target proximal anatomy, and may be particularly advantageous in procedures where the implantation of an annuloplasty ring 2050 is not indicated or desired, or in transcatheter delivery applications where the manifold anchor 2100 may be deployed through a catheter system without requiring open surgical access to the annulus.

In various embodiments, the manifold anchors 2100 may take any of a variety of forms known in the art, including but not limited to helical coil anchors, barbed anchors, nitinol tissue anchors, toggle bolt anchors, or clip-based anchors. The number, placement, and configuration of the manifold anchors 2100 may be selected based on the geometry of the suture manifold 100, the anatomy of the target fastening site, and the delivery mechanism employed, and may be determined by one of ordinary skill in the art. In some embodiments, where multiple manifold anchors 2100 are used, they may be distributed along the longitudinal axis LA of the suture manifold to provide uniform fixation across its full length.

With reference now to FIG. 12B, as discussed above, in certain embodiments, the suture manifold 100 may comprise one or more inlets 110, internal passages 120, and outlets 115 dedicated exclusively to the anchoring mechanism of the prosthesis 2000, i.e., the manifold anchors 2100. Accordingly, one or more anchor sutures 2150 may be configured to engage and tighten the manifold anchors 2100 against the local tissue, thereby securing the suture manifold 100 in its intended position at the repair site in the atrium. In the same or other embodiments, these components may be separate and distinct from the inlet(s) 110, internal passage(s) 120, and outlets 115 through which the repair sutures, i.e., the vertical sutures 155, are routed. This may be accomplished by any of the internal wall configurations described above. Thus, in some embodiments, the anchor sutures 2150 and the repair sutures 155 are physically separated from one another within the body 105 of the suture manifold 100 and do not interfere with one another during deployment and/or adjustment. Advantageously, the anchor sutures 2150 may be advanced, tensioned, and secured independently of the vertical repair sutures 155.

### Suture Management Device

In some embodiments, the heart valve prosthesis 2000 may further comprise a suture management device 165 through which the upper bundle 112 passes proximal to the inlet 110 of the suture manifold 100. The suture management device 165 may be positioned proximally to the inlet 110 and may be configured to create and/or receive the upper bundle 112 and facilitate the final adjustment and permanent fixation of the lengths of the vertical suture 155.

In some embodiments, the suture management device further includes a locking mechanism 166 and a cutting mechanism 167. The locking mechanism 166 may be configured to permanently fix the length of each vertical suture 155 by tying or otherwise fastening the upper bundle 112 at the desired suture length, thereby locking the tautness of the suture net 150 in its final adjusted configuration. The cutting mechanism 167 may be configured to sever any excess suture length remaining proximal to the fastening point, eliminating suture tails that could otherwise interfere with surrounding cardiac structures. The structure, function, and operational role of the suture management device 165 in the context of the heart valve prosthesis 2000 are substantially the same as described in the section directed to the suture management device 165 above.

As seen in FIG. 12B, in those embodiments of the prosthesis 2000 comprising anchoring sutures 2150, the anchoring sutures 2150 may be permanently fixed and trimmed by their own suture management device 165B in a manner similar to that of the suture management device 165A for the vertical sutures 155.

### Overview of the Delivery Catheter

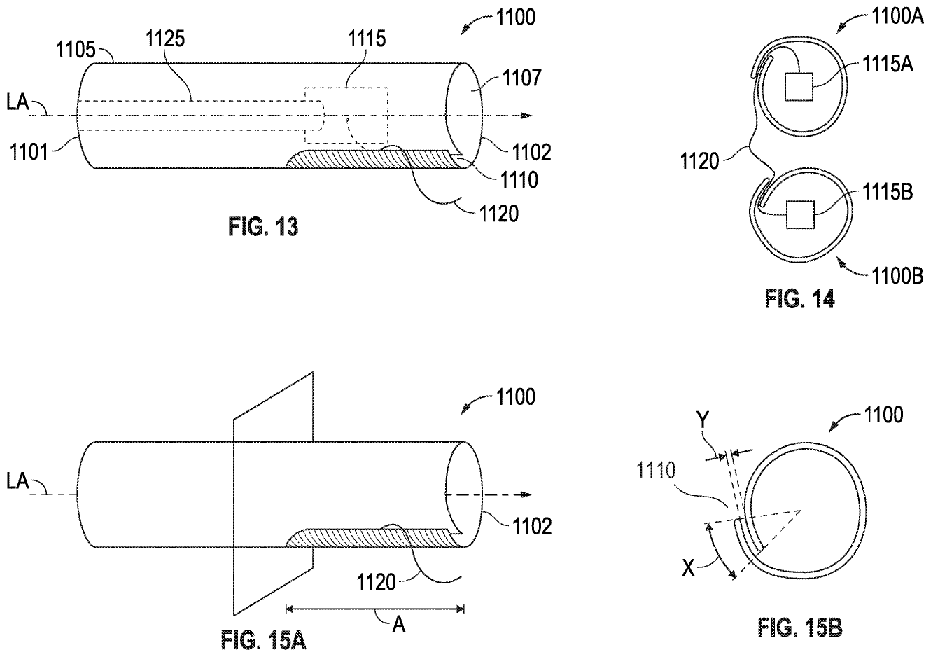

Turning to FIG. 13, there is shown a delivery catheter 1100 comprising an extruded body 1105, a longitudinal slit 1110, a transport element 1115 stored within the extruded body 1105, and a filament 1120 connected to the transport element 1115 and passing through the longitudinal slit 1110. In some embodiments, the delivery catheter 1100 may further comprise a transport element pusher 1125. In some embodiments, the delivery catheter 1100 may be cylindrical in shape or elliptic cylindrical in shape.

The delivery catheter 1100 may be configured to store and deliver a transport element 1115 to a target anatomical site, for example, during a transcatheter procedure. In some embodiments, the delivery catheter 1100 may be housed within a larger delivery sheath 2010 (not shown). In the same or other embodiments, the delivery catheter 1100 may be stored in the sheath 2010 (not shown) with one or more additional delivery catheters 1100 (not shown), transport elements 1115, prostheses, and/or other components for use in a transcatheter procedure. Together, these elements may form a unified prosthesis delivery system 2005 (not shown), discussed in greater detail below.

It may be appreciated, however, that the delivery catheter 1100 may also be used independently, without a surrounding delivery sheath 2010, for example, in procedures where delivery of a single catheter is sufficient.

With continued reference to FIG. 13, transport element 1115 may be stored within a lumen 1107 of the delivery catheter 1100 during advancement to the target anatomy. The transport element 1115 may itself be a prosthesis, or it may be a tool or intermediate component used to facilitate the installation of a prosthesis. In some embodiments, the transport element 1115 may be a ventricular anchor, a manifold anchor, or other implantable component of a prosthesis system delivery system 2005.

### Transport Element Pusher of the Delivery Catheter

With continued reference to FIG. 13, in some embodiments, the delivery catheter 1100 further comprises a transport element pusher 1125 housed within the body 1105 and proximal to the transport element 1115 along a longitudinal axis of the catheter 1100. The transport element pusher 1125 may be configured to apply a distal force to the transport element 1115.

The transport element pusher 1125 may be an elongate element stored within the lumen 1107 of the delivery catheter 1100. The transport element pusher 1125 may be substantially incompressible along its longitudinal axis LA, such that a proximally applied force (e.g. applied at a proximal end of the pusher 1125) is transmitted directly to the distal end of the pusher 1125 and thereby to transport element 1115. In some embodiments, the transport element pusher 1125 may enable precise and controlled distal advancement of the transport element 1115 through the catheter lumen 1107 and toward a target site.

The transport element pusher 1125 may take any of a variety of forms known to those of ordinary skill in the art. In some embodiments, the transport element pusher 1125 is a solid or hollow pushrod constructed from a stainless steel hypotube or nitinol tube. In some embodiments, the pusher 1125 is sufficiently flexible to navigate the curvature of the vasculature in a transcatheter procedure. In such embodiments, the pusher 1125 may be constructed, for example, of a braided or coiled wire. In the same or other embodiments, the pusher 1125 may exhibit a flexibility suitable for delivery through tortuous anatomical pathways, such as the atrial septum or aortic arch. The selection among these and other possible configurations may be informed by the dimensions and deployment force requirements of the transport element 1115, the length and curvature of the delivery path, and the overall design of the delivery system 2005.

### Filament of the Delivery Catheter

Turning to FIG. 14, in some embodiments, the filament 1120 may connect a first transport element 1115A housed within a first delivery catheter 1110A to a second transport element 1115B housed within a second delivery catheter 1100B. While two delivery catheters 1100A, 1100B are shown, in other embodiments, three or four or any plurality of delivery catheters 1100 are also possible and envisioned. Advantageously, multiple delivery catheters 1100 may facilitate independent actuation of multiple transport elements 1115. For example, multiple delivery catheters 1100 may facilitate coordinated and/or sequential actuation of multiple transport elements 1115.

In the same or other embodiments, the filament 1120 may connect the transport element 1115 to a prosthesis 2000 (not shown), as discussed in greater detail in the sections below.

In some embodiments, the filament 1120 may be a suture or other flexible elongate element suitable for tethering and/or for transmitting tension between the transport element 1115 and its connected counterpart(s). In some embodiments, actuation of multiple transport elements 1115 might be accomplished through a single continuous filament 1120, as described in greater detail in the sections below.

### Circumferential Slit of the Delivery Catheter

With reference to FIG. 15A, the longitudinal slit 1110 may extend along a portion of the extruded body 1105 and may allow the filament 1120 to exit the catheter 1100 laterally rather than through a distal end 1102. Advantageously, because the filament 1120 can exit through the slit 1110 rather than the opening at the distal end 1102, the transport element 1125 and the structure to which the filament 1120 connects—whether a prosthesis, a second transport element, or otherwise—may travel in substantially parallel paths within a prosthesis delivery system 2005 (not shown), and without the filament 1120 crossing between them in a manner that would cause tangling or interference. The slit 1110 may thereby maintain the organizational integrity of the filament 1120 routing throughout advancement and deployment of the delivery catheter 1100, which may be particularly important in configurations involving multiple catheters 1100 and/or multiple filaments 1120 traveling in close proximity within a delivery sheath 2010 (not shown).

In some embodiments, the slit 1110 may be sized to retain the transport element 1115 within the lumen 1107 of the catheter 1110, such that the transport element 1115 cannot be inadvertently expelled through the slit 1110 during advancement. Only the filament 1120 may pass through the slit 1110, while the transport element 1115 remains housed within the catheter 1100 until deliberately advanced by the transport element pusher 1125. In some embodiments, the slit 1110 and the distal end 1102 of the extruded body 1105 are contiguous, such that the filament 1120 may transition smoothly from exiting through the slit 1110 to exiting through the distal tip as the transport element 1115 is distally deployed at the target site.

At least some embodiments disclosed herein reflect the realization that the longitudinal slit 1110 in the delivery catheter 1100 may permit the filament 1120 to exit the catheter 1100 laterally along its length, while the transport element 1115 remains housed within the catheter 1100 until deliberately advanced. This configuration may allow the filament 1120 to be repositioned along the longitudinal axis LA of the catheter 1100 without the filament 1120 needing to be withdrawn proximally through the entire length of the catheter lumen 1107, thereby reducing the risk that the filament 1120 becomes tangled with adjacent filaments, sutures, or catheters housed within the same delivery sheath 2010.

In some embodiments, a length of the delivery catheter 1100 along its longitudinal axis LA may be substantially equal to that of a delivery sheath 2010 (not shown) and/or may be configured as appropriate for a transcatheter procedure. In the same or other embodiments, the delivery catheter 1100 may have a length between about 30 cm and about 250 cm, between about 50 cm and about 200 cm, between about 70 cm and about 150 cm, between about 80 cm and about 120 cm, or between about 90 cm and about 110 cm.

In some embodiments, the delivery sheath 2010 may have an outer diameter of between about 2 mm to about 20 mm (about 6 Fr to about 60 Fr), between about 4 mm to about 15 mm (about 12 Fr to about 45 Fr), between about 5 mm to about 10 mm (about 15 Fr to about 30 Fr), between about 6 mm to about 9 mm (about 18 Fr to about 27 Fr), or between about 7 mm to about 8 mm (about 21 Fr to about 24 Fr). As used herein, “Fr” or “French” refers to the French catheter scale, where one French unit corresponds to approximately one-third of a millimeter of outer diameter.

With reference to FIG. 15A, in some embodiments, the slit 1110 may have a length A in a direction of the longitudinal axis LA of the catheter 1100, said length A configured to accommodate the range of longitudinal positions the filament 1120 may occupy and traverse as the transport element 1115 is advanced and deployed, but short enough to preserve the structural integrity of the body 1105. In some embodiments, the slit 1110 may terminate at the distal end 1102 of the body 1105. In some embodiments, the slit 1110 may have a length A of at least 10 mm, at least 20 mm, between about 1 mm to about 500 mm, between about 1 mm to about 300 mm, between about 1 mm to about 100 mm, between about 10 mm to about 500 mm, between about 15 mm to about 300 mm, between about 20 mm to about 200 mm, or between about 50 mm to about 100 mm.

With reference to FIG. 15B, a cross-sectional view of the delivery catheter 1100 and the longitudinal slit 1110 is provided. In some embodiments, the slit 1110 may be wide enough to allow a filament 1120, such as a suture, to pass through freely, while being narrow enough to prevent inadvertent passage of the transport element 1115. In some embodiments, the slit 1110 may have a width Y of between about 0.2 mm and 2 mm, between about 0.25 mm to about 1.5 mm, between about 0.3 mm to about 1.0 mm, or between about 0.4 mm to about 0.7 mm.

With continued reference to FIG. 15B, in some embodiments, a circumferential overlap X refers to the angular extent that the slit 1110 overlaps around the circumference of the substantially cylindrical body 1105. A greater overlap X may allow the filament 1120 greater freedom of circumferential movement, while a lesser may better preserve the hoop strength and structural integrity of the catheter body 1105. In some embodiments, the circumferential overlap X may be between about 0 degrees to about 180 degrees, between about 5 degrees to about 135 degrees, between about 10 degrees to about 90 degrees, between about 15 degrees to about 75 degrees, between about 5 degrees to about 60 degrees, between about 5 degrees to about 30 degrees, or between about 10 degrees to about 25 degrees.

In some embodiments, the slit 1110 may have no circumferential overlap, equivalent to X being about 0 degrees or even a “negative overlap,” equivalent to X being negative degrees. The overlap may extend in either direction based on a circumferential point from which the angular measurement is taken; thus, an overlapping portion of the slit can extend counterclockwise or clockwise over an overlapped portion of the slit.

The foregoing dimensions are provided by way of example. The appropriate dimensions of the delivery catheter 1100 and its longitudinal slit 1110 may vary depending on a number of factors specific to the clinical application and the design of a prosthesis delivery system 2005, including the nature and dimensions of the transport element(s) 1115, the number of delivery catheters 1100, the specific transcatheter approach employed, and the gauge and material of the filament 1120 or suture passing through the slit 1110.

### Overview of the Prosthesis Delivery System

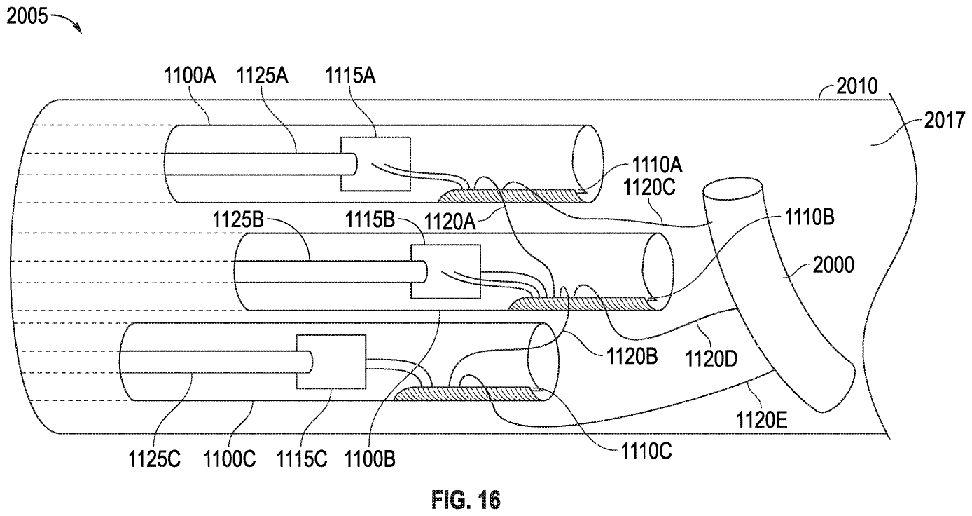

Turning to FIG. 16, there is shown a prosthesis delivery system 2005 comprising a delivery sheath 2010, a prosthesis device 2000 housed within the delivery sheath 2010, and a plurality of delivery catheters 1100 housed within the delivery sheath 2010. In some embodiments, each delivery catheter 1100 further comprises a longitudinal slit 1110, a housing a transport element 1115, and a transport element pusher 1125.

The prosthesis delivery system 2005 may be a catheter-based delivery platform configured to deliver, deploy, and install a heart valve prosthesis 2000 and all associated components to the target anatomy through a transcatheter approach. The prosthesis delivery system 2005 may be configured for a variety of transcatheter approaches known in the art, including but not limited to a transfemoral delivery, transjugular delivery, transseptal delivery, transapical delivery, transaortic delivery, among other possible procedures known to those in the art.

In some embodiments, all components necessary to complete the valve repair procedure may be housed within the delivery sheath 2010 simultaneously, allowing the entire procedure to be performed via a single percutaneous access point without the need to exchange or re-introduce delivery hardware between steps of the procedure. These components may include but are not limited to the prosthesis device 2000, its anchoring elements, deployment tools, or other intermediate components required during the procedure.

At least some embodiments disclosed herein reflect the realization that housing the prosthesis device 2000 and the plurality of delivery catheters 1100 within a common delivery sheath 2010 may enable the entire prosthesis delivery system 2005 to be advanced to a treatment site through a single transcatheter access point. This single-sheath integration may reduce the procedural complexity and the number of separate punctures or incisions required to deliver the components of the prosthesis 2000, may simplify navigation through the vasculature, and may reduce the risk of tangling among the multiple sutures, catheters, and ancillary components that would otherwise need to be advanced separately.

While in certain embodiments all transport elements 1115 and their respective delivery catheters 1100 are loaded into the delivery sheath 2010 prior to introduction into the anatomy and remain housed therein throughout the procedure, in other embodiments, not all components of the prosthesis delivery system 2005 need be housed within the delivery sheath 2010 simultaneously at any given point during the procedure. For example, the components of the delivery system 2005 may be introduced, deployed, and withdrawn sequentially, such that only a subset of the total complement of components occupies the delivery sheath 2010 at any one time. For example, a first delivery catheter 1100A housing a first transport element 1115A may be advanced through the delivery sheath 2010 to the target anatomy, the first transport element 1115A deployed, and the first delivery catheter 1100A then fully withdrawn from the delivery sheath 2010 before a second pusher catheter 1100B housing a second transport element 1115B is introduced into the sheath 2010 and advanced to its own respective target.

With continued reference to FIG. 16, individual transport elements 1115 may be housed within their own dedicated delivery catheter 1110 or may be stored directly within the lumen 2017 of the delivery sheath 2010 without an enclosing catheter 1110—referred to herein as a “naked” configuration. Housing a transport element 1115 within its own dedicated delivery catheter 1100 may provide independent and precise control over that element's advancement, positioning, and deployment, as the delivery catheter 1100 physically isolates the transport element 1115 from adjacent components. By contrast, a component stored in the naked configuration within the delivery sheath 2010 may be advanced or deployed by a shared mechanism or by the collective action of the components surrounding it.

In some embodiments, the prosthesis 2000, such as the heart valve prosthesis 2000 discussed in earlier sections, may be stored in the naked configuration within the delivery sheath 2010.

### Filament Routing Between Delivery Catheters

With continued reference to FIG. 16, three delivery catheters 1100A, 1100B, 1100C are shown. It should be appreciated that a different number of delivery catheters 1100 are also possible and envisioned, such as just one, or two, four, five, six, etc. Each delivery catheter 1100 may include a corresponding transport element 1115 and corresponding transport element pusher 1125.

In some embodiments, such as the one shown in FIG. 16, a first filament 1120A may run from a first transport element 1115A housed within a first delivery catheter 1100A, and through a first longitudinal slit 1110A of the first delivery catheter 1100A. In the same or other embodiments, the filament 1120A may further pass from the first longitudinal slit 1110A through a second longitudinal slit 1110B of a second delivery catheter 1100B, and connect to a second transport element 1115B housed within the second delivery catheter 1110B. Thus, a filament 1120A may connect a first transport element 1115A to a second transport element 1115B.

With continued reference to FIG. 16, in other embodiments, a second filament 1120B may similarly connect a second transport element 1115B to a third transport element 1115C. And in yet other embodiments, a filament 1120 might connect a third transport element to a fourth transport element, a fourth transport element to a fifth transport element, and so on. More generally, a filament 1120 may connect any two transport elements 1115 of the total plurality of transport elements 1115 housed within the delivery system 2005. In each case, the filament 1120 may travel between the respective longitudinal slits 1110 of the corresponding delivery catheters 1100.

In further embodiments, a single continuous filament 1120 may connect three or more transport elements 1115 simultaneously. For example, in FIG. 16, filament 1120A may be the same filament as 1120B. In this manner, a single filament 1120 may connect any combination of transport elements 1115 within the plurality thereof. In the same or other embodiments, this configuration might enable a single tensioning or advancement action applied to one end of the filament 1120 to propagate across multiple transport elements 1115 simultaneously or in sequence.

The filament routing configuration of the prosthesis delivery system 2005, including the specific combination of transport elements 1115 connected by any given filament 1120, may be selected based on the procedural requirements of the valve repair procedure and the desired deployment sequence.

### Filament Routing Between Delivery Catheters and the Prosthesis

In still further embodiments, the filament 1120 may connect one or more transport elements 1115 directly to the prosthesis device 2000 rather than—or in addition to—another transport element 1115. In FIG. 16, for example, a filament 1120C may run from the transport element 1115A through the longitudinal slit 1110A of its delivery catheter 1100A and connect to a designated attachment point on the prosthesis device 2000.

With continued reference to FIG. 16, in the same or other embodiments, multiple transport elements 1115A, 1115B, 1115C may each be connected by a respective filament 1120C, 1120D, 1120E to the prosthesis device 2000, which may provide independent control over each interaction between the corresponding transport element 1115 and prosthesis device 2000. Alternatively, multiple transport elements 1115 may be connected to the prosthesis device 2000 by a single shared filament 1120. In FIG. 16, for example, filament 1120C, 1120D, 1120E may be contiguous.

### Arrangement of Delivery Catheters Within the Delivery Sheath

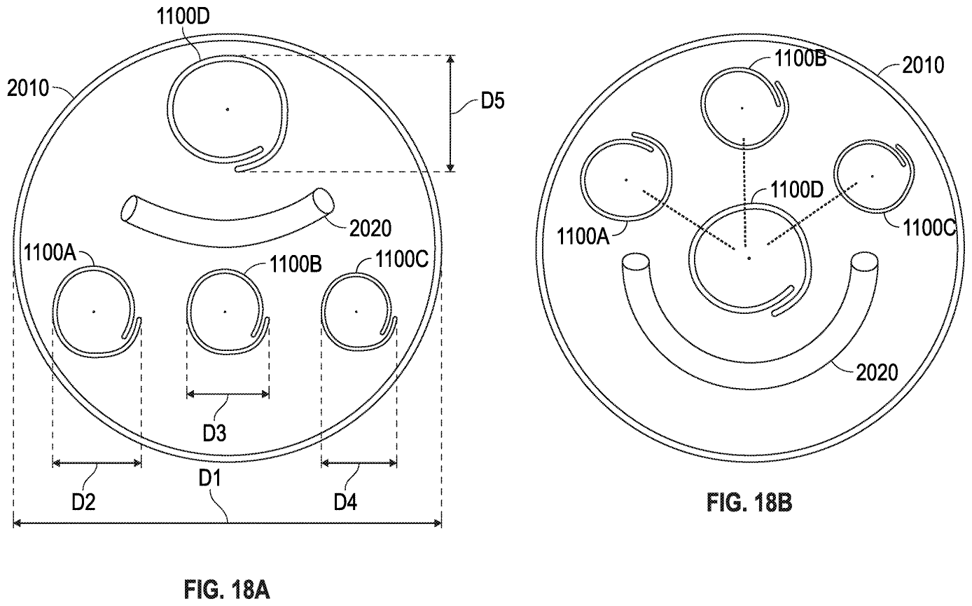

Turning now to FIGS. 18A and 18B, there are shown possible cross-sectional views of the delivery sheath 2010 housing a plurality of delivery catheters 1100.

In certain embodiments, two or more catheters 1100 may house analogous or identical transport elements 1115—for example, catheters 1100A, 1100B, 1100C shown in FIGS. 18A and 18B—and such catheters 1100A 1100B, 1100C may be identical or substantially identical to one another in diameter and construction.

In some embodiments, some or all of the delivery catheters 1100 are arranged substantially radially symmetrically about the central longitudinal axis LA of the delivery sheath 2010. In the same or other embodiments, the delivery catheters 1100 arranged symmetrically may be identical or substantially identical in diameter and/or share the same function, such as housing analogous or identical transport elements 1115.

With continued reference to FIGS. 18A and 18B, in some embodiments, one or more catheters 1100D may house a distinct transport element 1115D that differs in type, size, or function from the transport elements 1115 housed in the other catheters 1100 within the sheath 2010. In the same or other embodiments, the one or more catheters 1100D housing a distinct transport element 1115D may differ in diameter from the remaining catheters 1110 and may not participate in the radially symmetric arrangement of the other catheters.

In some embodiments, such as the one shown in FIG. 18B, one catheter 1100D may be arranged substantially coaxially with the longitudinal axis LA of the delivery sheath 2010, occupying the central position within the lumen 2017, while the remaining catheters 1100 are arranged substantially radially symmetrically about the same longitudinal axis LA.

In some embodiments, the diameter of the catheters 1100 may respectively reflect their different transport elements 1115 and/or functional roles within the prosthesis delivery system 2005. In some embodiments, the coaxial versus the radially arranged catheters 1100 may respectively reflect their different transport elements 1115 and/or functional roles within the prosthesis delivery system 2005.

### Heart Valve Prosthesis

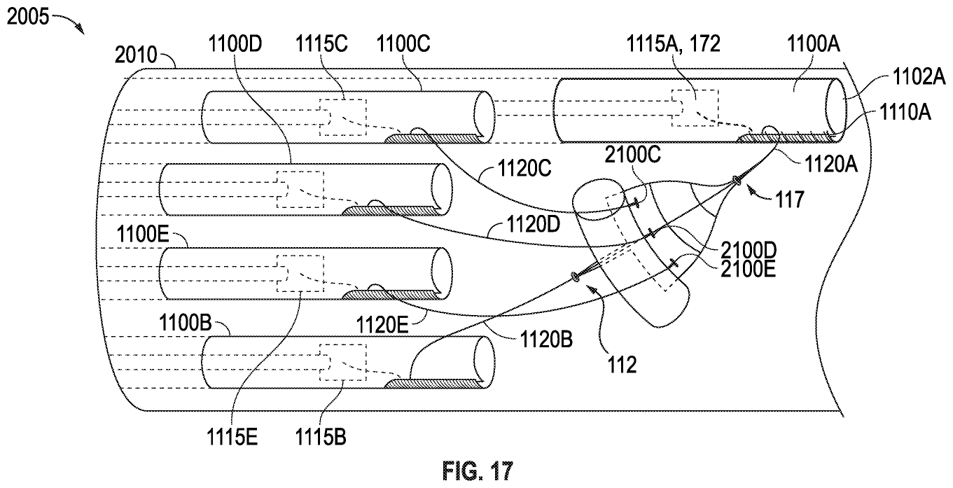

Turning now to FIG. 17, another exemplary embodiment of the prosthesis delivery system 2005 is shown. The prosthesis delivery system 2005 may house the heart valve prosthesis 2000 described in detail in the above sections, comprising at least the suture manifold 100, the suture net 150, and vertical sutures 155. In some embodiments the prosthesis 2000 may be housed together with all of the anchoring and deployment components required to complete the valve repair procedure, all within a single delivery sheath 2010 configured for transcatheter delivery to the target anatomy.

In the embodiment shown, the prosthesis 2000 may be housed in the naked configuration within the delivery sheath 2010. In some embodiments, the suture net 150 may be stored in a collapsed or compressed configuration. In the same or other embodiments, the vertical sutures 155 may be routed through their respective inlet(s) 110, internal passage(s) 120, and outlets 115 of the suture manifold 100. In the same or other embodiments, the prosthesis 2000 is pre-assembled within the delivery sheath 2010 prior to introduction into the patient. In some embodiments, the prosthesis 2000 may be housed in a dedicated delivery catheter 1100 within the delivery sheath 2010.

In the embodiment shown in FIG. 17, a first transport element 1115A may be the ventricular anchor 172, and may be housed within a first delivery catheter 1100A.

Accordingly, the first delivery catheter 1100A may be appropriately sized and positioned within the sheath 2010. For example, in some embodiments, the first delivery catheter 1100A may be arranged coaxially with the delivery sheath 2010.

The first transport element 1115A may be connected via a filament 1120A to the lower bundle 117 formed by the consolidated lower ends of the vertical sutures 155. In some embodiments, the filament 1120A might be the same as the lower bundle 117. For example, the lower bundle 117 may pass through the longitudinal slit 1110A of the first delivery catheter 1100A and be connected to the first transport element 1115A—the ventricular anchor 172—stored within the first delivery catheter 1100A. In the same or other embodiments, the first delivery catheter 1100A may be configured to deploy the ventricular anchor 172 at the target anatomy. During deployment, the first transport element pusher 1125A may advance the ventricular anchor 172 distally out of the first delivery catheter 1100A and into the ventricular anatomy, whereupon the anchor 172 is fixed to the target anatomy at the lower anchor point LAP. The lower bundle 117, traveling through either the longitudinal slit 1110A or the distal end 1102A of the delivery catheter 1100A, connects the ventricular anchor 172 to the lower ends of the vertical sutures 155 of the suture net 150.

With continued reference to FIG. 17, in the same or other embodiments, the vertical sutures 155 of the suture net 150 travel proximally from the lower bundle 117, form the suture net 150, separate and individually travel through the outlets 115, internal passage 120, and inlet 110 of the suture manifold 100, whereupon they are consolidated at their upper ends into the upper bundle 112, as described in detail in the sections above. In some embodiments, the upper bundle 112 in turn may serve as the filament 1120B connected to a second transport element 1115B of a second delivery catheter 1100B. In the same or other embodiments, the upper end of the sutures pass through a second longitudinal slit 1110B of the second delivery catheter 1100B.

In some embodiments, the second transport element 1115B may be the suture management device 165 described in the foregoing sections. In the same or other embodiments, the second transport element 1115 may be the suture management device 165 further comprising the fastening device 166 and the cutting device 167.

For example, during deployment, the upper bundle 112 may pass through the suture management device 165, which has been advanced to its intended position at the upper anchor point UAP, whereupon the suture management device 165 locks and trims the upper bundle 112 to permanently fix the tautness and geometry of the suture net 150.

As with the first delivery catheter 1100A, the second delivery catheter 1100B may be appropriately sized and positioned within the sheath 2010. In some embodiments, the second delivery catheter 1100B (as opposed to the first delivery catheter 1100A) may be arranged coaxially with the delivery sheath 2010.

With continued reference to FIG. 17, the filament 1120 of the delivery catheter 1100 may be connected to the prosthesis 2000. In some embodiments, the transport element 1115 may act on the prosthesis 2000 through the filament 1115, such as by tying or anchoring the prosthesis 2000. For example, a plurality of additional transport elements—shown here as 1115C, 1115D, 1115E—may be configured to secure the suture manifold 150 to the atrial anatomy. Each transport element 1115C, 1115D, 1115E may be housed within their respective delivery catheters 1100C, 1100D, 1100E.

In some embodiments, these delivery catheters 1100C, 1100D, 1100E may be substantially identical to one another in diameter and construction. In the same or other embodiments, these delivery catheters 1100C, 1100D, 1100E may be arranged radially symmetrically within the delivery sheath 2010. It may be appreciated that, while three delivery catheters 1100C, 1100D, 1100E are shown, a different number thereof is also possible and envisioned.

In one embodiment, each of delivery catheters 1100C, 1100D, 1100E (and others where applicable) may comprise a transport element 1115C, 1115D, 1115E and corresponding filament 1120C, 1120D, 1120E. Each filament 1120C, 1120D, 1120E may be configured to fix a suture manifold anchor 2100 of the suture manifold 100. As described in the foregoing sections, the suture manifold anchors 2100 may be configured to secure the suture manifold 100 to the target atrial anatomy—which may include the mitral annulus, the mitral leaflet, or the atrial wall—and may be implemented using any of a variety of anchoring technologies known to those of ordinary skill in the art. In some embodiments, the specific anchoring technology employed may determine the nature of the corresponding transport element 1115, which may serve as the installation device for that particular anchor 2100. In the same or other embodiments, the specific anchoring technology employed may also determine the nature of the corresponding filament 1120, which may connect the transport element 1115 to the installed anchor 2100.

In one example, the suture manifold anchor 2100 comprises a helical coil anchor of the type commonly used in transcatheter mitral valve repair procedures. The helical coil anchor may be configured to be screwed into the fibrous tissue of the mitral annulus or atrial wall by rotation of the anchor about its longitudinal axis. In the same or other embodiments, the corresponding transport element 1115 may be a rotational deployment device configured to engage the proximal end of the helical coil anchor and impart controlled rotational torque to advance the anchor 2100 into the target tissue. In the same or other embodiments, the filament 1120C, 1120D, 1120E may be a suture attached to the deployed helical coil anchor, passing through the longitudinal slit 1110C, 1110D, 1110E of the corresponding delivery catheter 1100C, 1100D, 1100E while said helical coil anchor is housed within the delivery catheter 1110 during delivery, and said filament 1120C, 1120D, 1120E connecting to the corresponding suture manifold anchor 2100 on the suture manifold 100.

In another example, the suture manifold anchor 2100 may comprise a barbed or toggling anchor, for example of a type used in transcatheter leaflet and annular fixation. In this embodiment, the anchor 2100 may be configured as a small dart or toggle element carried on the tip of a needle deployment device, which passes the anchor 2100 through the target tissue and releases it, such that the barbs or toggle mechanism engage the far surface of the tissue, preventing retraction. The corresponding transport element 1115 may be the needle deployment device itself, which is advanced through its delivery catheter 1100 and actuated, by the corresponding transport element pusher 1125, to drive the anchor 2100 through the tissue at the designated fixation point. Accordingly, while stored within the sheath 2010, the filament 1120C, 1120D, 1120E may be attached to the anchor 2100 and pass through the corresponding longitudinal slit 1110C, 1110D, 1110E of its corresponding delivery catheter 1100C, 1100D, 1100E.

In each of the foregoing examples, the transport element 1115 may be housed within its dedicated delivery catheter 1100 prior to deployment and may be advanced to the target tissue under operator control via the transport element pusher 1125. Following deployment of the transport element 1115 and fixation of the respective anchor 2100, the delivery catheter 1100 carrying the transport element 1115 may be retracted proximally and withdrawn from the delivery sheath 2010, leaving the filament 1120 in place to connect the deployed anchor 2100 and the suture manifold 100 and local anatomy.

In some embodiments, the fixation of the suture manifold anchors 2100 may be deployed sequentially. In the same or other embodiments, each suture manifold anchor 2100 may be connected via its own filament 1120, allowing each anchor 2100 to be independently advanced and deployed. In the same or other embodiments, each suture manifold anchor 2100 may be fixed via a shared filament 1120, allowing all of the anchors 2100 to be tensioned simultaneously.

In some embodiments, each delivery catheter 1100 associated with a suture manifold anchor 2100 may further comprise a dedicated cutting device (not shown) configured to sever the filament 1120 following fixation of the anchor 2100.

### Dimensions and Materials of the Delivery Sheath and Delivery Catheters

The delivery sheath 2010 may be sized to accommodate all components of the prosthesis delivery system 2005 simultaneously within its lumen 2017, including the prosthesis device 2000 and the full complement of catheters 1100, while remaining within the dimensional constraints imposed by the given transcatheter delivery route.

With respect to longitudinal length, the delivery sheath 2010 and the catheters 1100 housed within it may be substantially similar in length, as each catheter 1100 must extend substantially the full working length of the delivery sheath 2010 in order for the transport element pusher 1125 to reach from the proximal handling mechanism outside the body to the transport element 1115 at the distal tip of the system 2000. Both the longitudinal length of the delivery sheath 2010 and its housed catheters 1110 may be primarily determined by the distance from the percutaneous access point to the target anatomy. In some embodiments, the transport element pusher 1125 may extend substantially the full length of the delivery catheter 1100. In some embodiments, the pusher 1125 may further extend out of the proximal end of the delivery catheter 1100, providing a mechanism through which the operator may apply a controlled distal force. In some embodiments, the operator uses the pusher 1125 to drive the transport element 1115 out of the distal end 1102 of the delivery catheter 1100 and deploys it at the target anatomical site.

With respect to diameter, in some embodiments the delivery sheath 2010 is large enough in its inner diameter to house the prosthesis device 2000 and all catheters 1100 simultaneously and substantially in parallel along the longitudinal axis LA, with sufficient clearance to allow each catheter 1100 to be advanced, retracted, and rotated independently. At the same time, the outer diameter of the delivery sheath 2010 may be small enough to be compatible with the chosen percutaneous access route. In some embodiments, the inner diameter of the delivery sheath may be governed by its relationship to the diameters of the catheters 1100 and the size of the prosthesis device 2000 housed within the sheath 2010; and/or the outer diameter of each delivery catheter 1100 may be governed by its relationship to the inner diameter of the delivery sheath 2010. In some embodiments, the sheath diameter may be sized to minimize empty space within the lumen 2017, reducing the freedom of movement of the housed components. In other embodiments, the sheath 2010 diameter may be sized to allow a degree of radial freedom of movement of the catheters 1100 within the lumen 2017, which may improve the flexibility of the fully loaded sheath and reduce the risk of kinking during advancement.

In some embodiments, the delivery sheath 2010 may have an outer diameter of between about 2 mm to about 20 mm, between about 4 mm to about 15 mm, between about 5 mm to about 10 mm, between about 6 mm to about 9 mm, or between about 7 mm to about 8 mm.

In some embodiments, each delivery catheter 1100 may have an outer diameter of between about 0.5 mm to about 5.0 mm, between about 0.8 mm to about 3.5 mm, between about 1.0 mm to about 2.5 mm, or between about 1.25 mm to about 2.0 mm.

In some embodiments, the delivery sheath 2010 of the prosthesis delivery system 2005 may be sufficiently flexible and steerable to navigate the anatomy of the transcatheter delivery route, including for example passage through the curvature of atrium/atrial septum, or the aortic arch. In some embodiments, the delivery sheath 2010 may be constructed from any biocompatible material or combination of materials suitable for intravascular use. For example, the delivery sheath may comprise a multi-layer wall construction including an inner liner constructed from polytetrafluoroethylene (PTFE) or expanded PTFE, a middle reinforcement layer comprising a braided or coiled structure of stainless steel or nitinol wire, and an outer jacket constructed from a biocompatible polymer such as polyether block amide (PEBA), polyurethane, or nylon.

In certain embodiments, the stiffness of the outer jacket may be varied along the longitudinal length of the sheath. In the same or other embodiments, the varied stiffness may provide a graduated flexibility profile that facilitates navigation through the tortuous anatomy of atrium-septum-ventricle or the aortic arch while maintaining incompressibility and force transfer at the proximal end.

In some embodiments, imaging guidance apparatus, such as radiopaque markers, may additionally be incorporated at the distal tip and/or along the length of the sheath. In some embodiments, a distal end of the delivery sheath 2010 is steerable in various directions and degrees in a controlled fashion.

### Overview of the Method of Surgical Use of Heart Valve Prosthesis

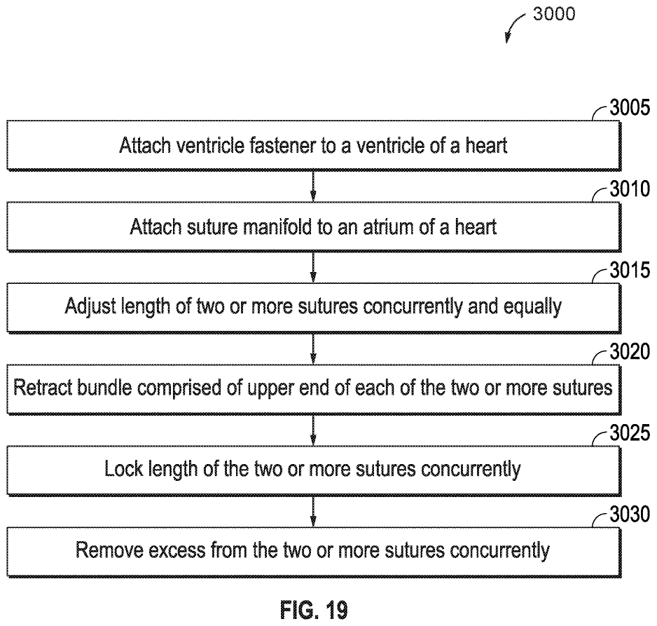

Turning to FIG. 19, there is shown a method 3000 of surgically repairing a heart valve comprising the steps of attaching a ventricle fastener to a ventricle of a heart (3005), attaching a suture manifold to an atrium of a heart (3010), and adjusting a length of two or more sutures, concurrently and equally (3015). In some embodiments, the step of adjusting the length of the two or more sutures (3015) may further include retracting a bundle comprised of an upper end of each of the two or more sutures (3020), locking the length of the two or more sutures concurrently (3025), and removing an excess from the two or more sutures concurrently (3030). In some embodiments, the two or more sutures directly or indirectly connect the ventricle fastener to the suture manifold. For example, the two or more sutures may connect at a lower end to the ventricle fastener and connect at an upper end to the suture manifold.

The two or more sutures may form a suture net and, together with the suture manifold, form the heart valve prosthesis described in the sections above. For example, method 3000 may describe a procedure for repairing an atrioventricular valve by establishing a subvalvular suture net and by adjusting, locking, and trimming all sutures simultaneously through a single operation. In preferred embodiments, the method 3000 may be performed through an open surgical approach. For example, the method 3000 may be performed in conjunction with a sternotomy, minimally-invasive mini-thoracotomy, video-assisted approach, or robotic approach, wherein the steps are carried out under direct visualization. Notwithstanding, method 3000 may also be carried out in a transcatheter approach, wherein the steps are carried out percutaneously using the prosthesis delivery system 2005 described above.

### Attachment of the Ventricle Fastener

With continued reference to FIG. 19, in some embodiments, the step of attaching the ventricle fastener (3005) may further comprise suturing a bundle comprising a lower end of each of the two or more sutures to a pledget and to the ventricular anatomy. In some embodiments, the ventricle fastener may be attached to the ventricular anatomy to establish the lower anchor point prior to suture length adjustment in the subsequent steps. In various embodiments, the ventricle fastener may be attached to the papillary muscle, the ventricular wall, or other local ventricular anatomy, and/or the specific attachment site may be selected based on the anatomy of the patient and the nature of the valve repair procedure being performed.

### Attachment of the Suture Manifold

The suture manifold may be attached to the atrial anatomy to establish the upper anchor point of the prosthesis. With continued reference to FIG. 19, in some embodiments, the step of attaching the suture manifold (3010) may involve directly attaching the suture manifold to the mitral annulus, the mitral leaflet, or the atrial wall via one or more manifold anchors. In this configuration, each manifold anchor is deployed directly into the target tissue and serves as the fixation element between the suture manifold and the native anatomy, as described in detail in the sections above. Notably, direct anchor attachment may be preferred in transcatheter approaches where annuloplasty ring implantation is not indicated or practicable, or in cases where the native annular tissue is of sufficient quality to support direct anchoring without the load-distributing benefit of a ring. With open surgical approach, the suture manifold 100 may be fixed to the mitral annulus, the mitral leaflet, the atrial wall, or a mitral annuloplasty ring by surgical sutures under direct or video-assisted visualization.

In some embodiments, the suture manifold may be positioned, such that the outlets of the manifold are directed toward lower anchor point at or near the ventricular anatomy.

Alternatively, in some embodiments, the step of attaching the suture manifold (3010) further comprises suturing an annuloplasty ring to the heart and attaching the suture manifold to the annuloplasty ring. This approach may be preferred in open surgical procedures, such as the present method 3000. The use of the annuloplasty ring may be preferred where the annuloplasty ring can be precisely positioned and sutured under direct visualization and/or where simultaneous annular remodeling may be a goal of the procedure. The annuloplasty ring may be a complete ring or a partial band, rigid or flexible, and may be selected and sized to correspond to the anatomy of the patient's valve annulus as described above.

### Adjustment of Suture Length

As described in detail in the foregoing sections, the suture manifold may comprise one or more inlets, internal passages, and outlets. The same descriptions and disclosures will likewise apply to the method 3000, wherein the one or more sutures may travel from the ventricle fastener and through one or more outlet(s), internal passage(s), and inlet(s) of the suture manifold.

In some embodiments, this routing path may be established during assembly of the prosthesis, for example preoperatively. In some embodiments where the suture manifold comprises multiple siloed internal passages, each suture may be routed through the passage dedicated to its functional group. For example, this may maintain separation between anchor sutures and repair sutures or between distinct subsets of repair suture. By routing each suture through the suture manifold, the method 3000 further ensures that the travel displacement and length of each suture are controlled.

With continued reference to FIG. 19, the method 3000 includes concurrent and equal adjustment of the lengths of the two or more sutures (3015). In some embodiments, adjustment of the lengths of the two or more sutures corresponds to an adjustment of a tautness of the entire suture net.

In some embodiments, the step of adjusting the length of two or more sutures (3015) may further comprise concurrently adjusting a bundle comprised of an upper end of each of the two or more sutures. For example, the upper bundle may consolidate the upper ends of all sutures proximal to the inlet of the suture manifold, such that a single proximal movement of the bundle draws all sutures equally and/or increases the tautness of the suture net uniformly. In the same or other embodiments, the concurrent adjustment may also be equal in length, as in each suture may be retracted or advanced the same length. In some embodiments, the operator may advance and retract the upper bundle iteratively, assessing suture net tautness and leaflet coaptation, until the desired net geometry and valve function are achieved.

In some embodiments, the total plurality of sutures may be divided into subsets, and each subset of sutures may be independently advanced or retracted. Accordingly, the method 3000 may further comprise adjusting a length of a second set of sutures concurrently (and in some embodiments equally) through retraction of a second bundle, independently of the first bundle and the first set of sutures. This may allow distinct regions of the heart valve prosthesis—such as sutures associated with distinct leaflets in a commissure operation—or distinct functional groups of sutures—such as anchor sutures and repair sutures—to be adjusted separately. In some embodiments, the distinct bundles may travel through separate and siloed internal passages of the suture manifold. In some embodiments, adjustment of the distinct bundles for each subset may be sequential and, in other embodiments, may be in parallel. In some embodiments, a tautness of a first subset of sutures may differ from a tautness of a second subset of sutures. It should be noted that a plurality of suture subsets, not just two, is also possible and envisioned.

With continued reference to FIG. 19, there is shown step 3025, wherein the length of the two or more sutures is locked concurrently. Once the desired suture lengths and net tautness have been achieved through retraction of the upper bundle, the length of all sutures is permanently and simultaneously locked, for example through a suture management device, ensuring that the adjusted geometry of the suture net is fixed and does not change meaningfully after the device is withdrawn. The step of locking the two or more sutures in method 3000, as described above, incorporates the locking mechanism 166 of the suture management device 165 described in detail in the context of the heart valve prosthesis 2000 above, and the reader is referred to that section. As described therein, the locking mechanism 166 is configured to permanently fix the length of each vertical suture by tying or otherwise fastening the upper bundle at or proximal to the upper anchor point, and may be implemented through any of the knotting, clipping, crimping, or integrated fastening techniques discussed above. In the context of method 3000, the locking mechanism 166 may be actuated following the adjustment step, after the desired suture lengths and net tautness have been achieved through retraction of the upper bundle, and its actuation permanently fixes the geometry of the suture net.

For example, in some embodiments, the step of locking further comprises crimping a sleeve onto the upper end of each suture. The crimp may mechanically deform the sleeve around the suture to permanently fix its length without the need for a tied knot.

With continued reference to FIG. 19, there is shown step 3030, wherein an excess is removed from the two or more sutures concurrently. Any suture length remaining proximal to the locking point may be severed simultaneously from all sutures, for example by using a cutting mechanism of the suture management device. The structure, function, and specific implementations of the cutting mechanism are described in full above and the reader is referred to that section accordingly.

In some embodiments, the cutting mechanism is integrated into the same suture management device as the locking mechanism. In the same or other embodiments, the step of locking and cutting are performed sequentially in a single device actuation without repositioning. In alternative embodiments, the cutting mechanism is a discrete element of the suture management device that is actuated independently of the locking mechanism. In either configuration, concurrent trimming of all sutures can eliminate suture tails that could otherwise contact surrounding cardiac structures and ensures a clean final suture configuration prior to withdrawal of the delivery system.

### Overview of the Method of Transcatheter Use of the Heart Valve Prosthesis

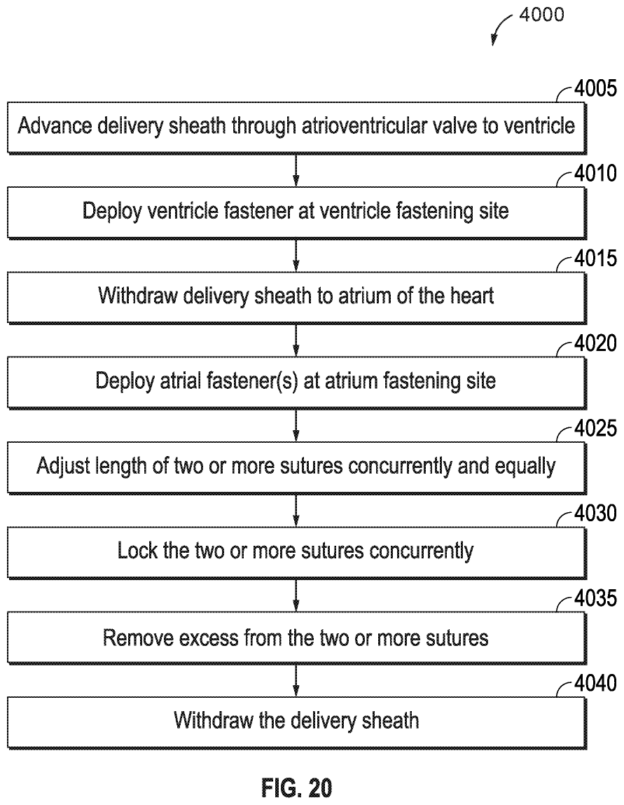

Turning to FIG. 20, there is shown a method 4000 of repairing a heart valve via transcatheter implant comprising the steps of: advancing a delivery sheath through an atrioventricular valve and to a ventricle of the heart (4005), the delivery sheath containing a ventricle fastener, a suture manifold, an atrial fastener, a suture management device, two or more sutures, and a plurality of catheters; deploying the ventricle fastener at a ventricle fastening site (step 4010); withdrawing the delivery sheath to an atrium of the heart (4015); deploying the atrial fastener at the atrium fastening site (4020); adjusting a length of the two or more sutures concurrently (4025); locking the two or more sutures concurrently (4030); removing an excess from the two or more sutures concurrently (4035); and withdrawing the delivery sheath (4040).

In contrast to method 3000, the method 4000 may provide a transcatheter procedure for delivering and installing the heart valve prosthesis described herein through a single delivery sheath, without the need for open surgical access.

In some embodiment all components necessary to complete the repair are pre-loaded within the delivery sheath prior to introduction. In some embodiments, imaging guidance, for example fluoroscopic or echocardiographic guidance, are utilized by the operator during the procedure.

### Transcatheter Use Delivery Approach

In some embodiments, the method (4000) is for a mitral valve repair, and the step of advancing the delivery sheath (4005) may further include advancing the sheath through a femoral vein, jugular vein, a femoral artery, or via a transapical approach. For example, the delivery sheath may be advanced via a transfemoral venous approach, wherein the sheath is introduced through the femoral vein, advanced through the inferior vena cava, into the right atrium, and across the interatrial septum into the left atrium. In some embodiments, the delivery sheath may be advanced via a jugular vein approach, wherein the sheath is introduced through the jugular vein, advanced through the superior vena cava, into the right atrium, and across the interatrial septum into the left atrium. In an alternative embodiment, a transfemoral arterial approach may be employed, wherein the sheath is introduced through the femoral artery, advanced retrograde through the aorta and aortic arch, and across the aortic valve into the left ventricle. In another alternative embodiment, specifically a transapical approach, the sheath may be introduced directly into the left ventricle through a small incision at the cardiac apex.

In some embodiments, the method (4000) is for a tricuspid valve repair, and the step of advancing the delivery sheath (4005) may further include advancing the delivery sheath through a femoral vein or a jugular vein. For example, the delivery sheath may be advanced via the femoral vein, through the inferior vena cava, and into the right atrium, from which the tricuspid valve is directly accessible without the need for transseptal puncture. In an alternative embodiment, the sheath may be introduced through the jugular vein and advanced through the superior vena cava into the right atrium.

### Deployment of the Ventricle Fastener

In some embodiments, the plurality of catheters includes a ventricle fastener catheter comprising a longitudinal slit and containing the ventricle fastener and a ventricle fastener pusher. In the same or other embodiments, the step of deploying the ventricle fastener (4010) may further include distally advancing the ventricle fastener pusher such that the ventricle fastener exits the ventricle fastener catheter, deploying the ventricle fastener, and thereafter withdrawing the ventricle fastener catheter.

Once the delivery sheath has been advanced to the ventricle fastening site, the ventricle fastener may be distally advanced within its dedicated delivery catheter via the ventricle fastener pusher, driving the ventricle fastener out of the distal end of the catheter and into contact with the ventricular anatomy at the fastening site. The longitudinal slit of the ventricle fastener catheter allows the filament connecting the ventricle fastener to travel alongside the catheter without tangling during advancement and deployment. In some embodiments, the filament is none other than the two or more sutures which form the suture net. Thus, before deployment, the two or more sutures may be connected to the ventricle fastener, pass through the longitudinal slit of the ventricle fastener catheter, and connect to and pass through the suture manifold. Once the ventricle fastener is confirmed to be securely deployed, the ventricle fastener catheter is withdrawn proximally from the delivery sheath, leaving the deployed fastener and its connected filament, i.e., the two or more sutures, in place.

Thereafter, the delivery sheath and its remaining housed components (less the ventricle fastener and connected filament) may be distally withdrawn to the next deployment site at the atrium of the heart (step 4015).

### Deployment of the Atrial Fastener

With continued reference to FIG. 20, the plurality of catheters may include at least one atrial fastener catheter, each atrial fastener catheter comprising a longitudinal slit and containing an atrial fastener and an atrial fastener pusher. In method 4000, the step of deploying the atrial fastener(s) (4020) may further comprise distally advancing the atrial fastener pusher such that the atrial fastener exits the atrial fastener catheter, and thereafter withdrawing the atrial fastener catheter.

After the delivery sheath has been withdrawn to the atrium, each atrial fastener catheter may be advanced to its designated fixation site on the mitral or tricuspid annulus, leaflet, or atrial wall, and the atrial fastener pusher may be distally advanced to deploy the atrial fastener into the target tissue.

In some embodiments, the step 4020 may further comprise fixing a suture manifold anchor to the atrium fastening site. For example, the manifold anchor may comprise a base element positioned on an external surface of the body of the suture manifold. During deployment, the base element may press against the target atrial tissue, the base element including one or more apertures through which a fixation element may be passed. The atrial fastener may function as a deployment device configured to drive a fixation element—such as a helical coil, a barbed pin, barbed needle, or a suture needle—through the aperture of the manifold anchor and into the underlying atrial tissue, pinning the manifold anchor flush against the tissue surface.

In some embodiments, once the fixation element is deployed and the manifold anchor is secured, the anchor suture is tensioned from the inlet of the suture manifold to draw the manifold into its final seated position. In certain embodiments, the anchor sutures connecting the manifold anchors to the suture manifold may be tensioned concurrently through a dedicated anchor bundle, formed by consolidating the upper ends of all anchor sutures proximal to their respective inlets in a manner analogous to the upper bundle of the repair sutures described above. The concurrent tensioning of the anchor sutures through a unified anchor bundle allows all manifold anchors to be tightened simultaneously, seating the suture manifold uniformly against the target atrial anatomy in a single tensioning operation. The mechanisms by which the anchor bundle is formed, tensioned, locked, and trimmed are substantially as described in the sections above, and the reader is referred to those sections accordingly.

In some embodiments, step 4020 further comprises repeating the same deployment steps for each atrial fastener catheter in sequence until all atrial fasteners have been deployed. Where there are multiple atrial fastener catheters, each may comprise a longitudinal slit and contain its own respective atrial fastener and respective atrial fastener pusher. In some embodiments, each catheter may be deployed sequentially, such that each atrial fastener is independently advanced, deployed, and confirmed. In other possible embodiments, each catheter may be deployed in parallel. In the same or other embodiments, each atrial fastener catheter may be deployed and tensioned iteratively.

### Adjusting the Length of the Repair Sutures

In some embodiments, the delivery sheath further comprises two or more sutures, each suture connecting at a lower end to the ventricle fastener, each suture passing through an outlet of the suture manifold, each suture passing through an inlet of the suture manifold, and each suture configured to connect at an upper end, directly or indirectly, to the atrial fastener.

The two or more sutures may be pre-loaded within the delivery sheath prior to introduction, already routed through their respective inlets, outlets, and internal passages of the suture manifold, such that the suture net is substantially pre-assembled within the delivery sheath in a compressed delivery configuration. Each suture connects at its lower end to the ventricle fastener and then travels proximally through a dedicated outlet and internal passage of the suture manifold to the inlet, where the upper ends are consolidated into the upper bundle.

With continued reference to FIG. 20, in step 4025, the step of adjusting the length may further comprise proximally withdrawing an upper bundle, the bundle being formed by connecting each suture at an upper end proximal to the inlet of the suture manifold. The upper bundle may be proximally withdrawn—which may be accomplished by the operator from outside the body—to simultaneously and equally retract all sutures and increase the tautness of the suture net, as described in detail in the sections above. For example, the operator may iteratively advance and retract the upper bundle while assessing valve function under imaging guidance until the desired net geometry and leaflet coaptation are achieved.

### Tightening Catheter and Suture Management Catheter

In certain embodiments, the delivery sheath further houses a tightening catheter containing a tightening transport element connected to the upper bundle of the two or more sutures. The tightening transport element may serve as the primary mechanism by which the operator adjusts suture length and net tautness during step 4025, allowing the upper bundle to be proximally withdrawn or distally advanced from outside the body by retracting or advancing the tightening transport element through its catheter via a transport element pusher. This configuration may unify suture length adjustment into a single operator-controlled element. Once the desired suture lengths have been achieved, the tightening transport element may be held in position while the suture management device locks and trims the sutures, as described below, after which the tightening catheter may be withdrawn from the delivery sheath.

Following adjustment of the suture lengths, the two or more sutures may be locked and trimmed by the suture management device (step 4030), which is positioned at the upper bundle proximal to the upper anchor point. The structure, function, and operational details of the suture management device are described in detail in the sections above, and the reader is referred thereto accordingly.

In one possible embodiment, the suture management device is housed within the same catheter as the tightening transport element, such that the tightening catheter serves the dual function of advancing the tightening transport element during the adjustment phase and delivering the suture management device during the locking and cutting phase.

In a second possible embodiment, the suture management device may be housed within its own dedicated suture management catheter, independent of the tightening catheter, such that the two catheters may be advanced, positioned, and actuated independently of one another. In this configuration, the tightening catheter may be withdrawn following completion of the adjustment phase before the suture management catheter is advanced to the upper bundle. Thereafter, the suture management device may be actuated. For example, the suture management device may be deployed to lock and/or cut the two or more sutures.

Upon completion of method 4000, including deployment of the ventricle fastener and atrial fasteners, adjustment of the suture lengths, locking of the upper bundle, and trimming of the excess suture, all remaining components of the delivery system may be withdrawn from the body. In some embodiments, one or more delivery catheters may be individually retracted into the delivery sheath before the sheath itself is withdrawn through the vasculature and out of the percutaneous access site (step 4040). Prior to full withdrawal, the operator may confirm under imaging guidance, such as through fluoroscopic or echocardiographic imaging, that the deployed prosthesis components remain in their intended positions and that the valve repair has achieved the desired functional result.

The percutaneous access site may then be closed by standard techniques known in the art, completing the transcatheter valve repair procedure.

### Illustration of Subject Technology As Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. A suture manifold comprising a body, a first inlet, a first internal passage, and a plurality of outlets, wherein the first inlet is connected to at least one of the plurality of outlets through the first internal passage.
Clause 2. The suture manifold of any of the foregoing embodiments, further comprising a second inlet and a second internal passage, wherein the second inlet is connected to at least one of the plurality of outlets through the second internal passage.
Clause 3. The suture manifold of any of the foregoing embodiments, wherein the first internal passage and the second internal passage are siloed from one another.
Clause 4. The suture manifold of any of the foregoing embodiments, further comprising a plurality of inlets and a plurality of internal passages, wherein each internal passage connects one of the inlets to at least one of the plurality of outlets, and wherein at least one internal passage of the plurality is siloed from at least one other internal passage of the plurality of internal passages.
Clause 5. The suture manifold of any of the foregoing embodiments, wherein the first internal passage and the second internal passage are siloed by a frontal internal wall or a transverse internal wall.
Clause 6. The suture manifold of any of the foregoing embodiments, wherein the first internal passage and the second internal passage are siloed by a sagittal internal wall.
Clause 7. The suture manifold of any of the foregoing embodiments, wherein each of the plurality of internal passages are siloed from one another by one or more radial internal walls.
Clause 8. The suture manifold of any of the foregoing embodiments, wherein the first inlet is located along a central plane of the body.
Clause 9. The suture manifold of any of the foregoing embodiments, wherein the first inlet is located off-center from a central plane of the body.
Clause 10. The suture manifold of any of the foregoing embodiments, wherein the plurality of outlets are located collinearly along the body.
Clause 11. The suture manifold of any of the foregoing embodiments, wherein each outlet of the plurality of outlets is equally spaced from one another with respect to a longitudinal axis of the body.
Clause 12. The suture manifold of any of the foregoing embodiments, wherein the body is cylindrical in shape.
Clause 13. The suture manifold of any of the foregoing embodiments, wherein the body is arcuate cylindrical in shape.
Clause 14. The suture manifold of any of the foregoing embodiments, wherein the body is bent cylindrical in shape.
Clause 15. The suture manifold of any of the foregoing embodiments, wherein one or more dimensions of the suture manifold are configured to correspond to a number of sutures passing through the body.
Clause 16. The suture manifold of any of the foregoing embodiments, wherein one or more dimensions of the suture manifold are configured to correspond to an atrioventricular valve annulus shape of a patient recipient.
Clause 17. The suture manifold of any of the foregoing embodiments, wherein one or more dimensions of the suture manifold are configured to correspond to a specific atrioventricular valve repair procedure.
Clause 18. The suture manifold of any of the foregoing embodiments, wherein the body is semi-rigid.
Clause 19. The suture manifold of any of the foregoing embodiments, wherein the body is constructed from polytetrafluoroethylene, polyethylene terephthalate, or polyurethane.
Clause 20. The suture manifold of any of the foregoing embodiments, wherein the body has a longitudinal length between about 10 mm to about 60 mm and a diameter between about 1 mm and 20 mm.
Clause 21. The suture manifold of any of the foregoing embodiments, wherein the body has a longitudinal length matching a full circumference of the valve annulus.
Clause 22. The suture manifold of any of the foregoing embodiments, wherein the body is part of a complete or incomplete annuloplasty ring.
Clause 23. An adjustable suture net comprising at least two vertical sutures, each vertical suture being fixed at an upper anchor point and a lower anchor point, and at least one horizontal thread tethering one or more vertical sutures, wherein a length of each vertical suture between the upper anchor point and the lower anchor point is adjustable; and the length of each vertical suture controls a tautness of each vertical suture.
Clause 24. The adjustable suture net of any of the foregoing embodiments, wherein the at least one horizontal thread comprises two or more horizontal threads spaced along a vertical axis.
Clause 25. The adjustable suture net of any of the foregoing embodiments, wherein a tautness of each vertical suture is substantially equal across the vertical sutures.
Clause 26. The adjustable suture net of any of the foregoing embodiments, wherein the length of each vertical suture is proportional to a travel displacement of each vertical suture between the upper anchor point and the lower anchor point, and in some embodiments, directly proportional to the travel displacement. Thus, the longer the suture, the greater the travel displacement or travel length.
Clause 27. The adjustable suture net of any of the foregoing embodiments, wherein a travel displacement of each vertical suture is equal.
Clause 28. The adjustable suture net of any of the foregoing embodiments, further comprising an upper bundle tethering together an upper end of each vertical suture and a lower bundle tethering together a lower end of each vertical suture.
Clause 29. The adjustable suture net of any of the foregoing embodiments, wherein a tautness of each vertical suture is maintained equal as each vertical suture is lengthened or shortened.
Clause 30. The adjustable suture net of any of the foregoing embodiments, wherein a ratio of a minimum travel length to suture length of each vertical suture is maintained as each vertical suture is lengthened or shortened.
Clause 31. The adjustable suture net of any of the foregoing embodiments, wherein the length of each vertical suture is adjusted by distally advancing or proximally withdrawing the upper bundle.
Clause 32. The adjustable suture net of any of the foregoing embodiments, further comprising a fastening device configured to adjust the length of each vertical suture.
Clause 33. The adjustable suture net of any of the foregoing embodiments, wherein the fastening device is configured to remove any excess of each suture proximal to the fastening device.
Clause 34. A heart valve prosthesis comprising: two or more vertical sutures, each suture having an upper end configured to be fastened to an atrium of a heart and a lower end configured to be fastened to a ventricle of the heart; and a suture manifold directing a path of each of the two or more vertical sutures; wherein the suture manifold controls a length of each suture.
Clause 35. The heart valve prosthesis of any of the foregoing embodiments, wherein the lower end of each suture is coupled into a lower bundle before being fastened to the ventricle of the heart.
Clause 36. The heart valve prosthesis of any of the foregoing embodiments, further comprising a net located below the suture manifold, formed by the two or more sutures and at least one horizontal thread connecting each suture.
Clause 37. The heart valve prosthesis of any of the foregoing embodiments, wherein a tautness of the net is controlled by the length of each suture.
Clause 38. The heart valve prosthesis of any of the foregoing embodiments, wherein a net density is characterized by a number of sutures, a number of horizontal threads, and a tautness of the net.
Clause 39. The heart valve prosthesis of any of the foregoing embodiments, wherein the net density is adjusted according to a specific atrioventricular valve repair procedure.
Clause 40. The heart valve prosthesis of any of the foregoing embodiments, wherein a tautness of each suture is maintained to be substantially equal by the suture manifold.
Clause 41. The heart valve prosthesis of any of the foregoing embodiments, wherein a ratio of the length of each suture to its minimum travel length is maintained to be equal by the suture manifold.
Clause 42. The heart valve prosthesis of any of the foregoing embodiments, wherein a tautness of each suture is maintained to be equal by the suture manifold.
Clause 43. The heart valve prosthesis of any of the foregoing embodiments, wherein the length of each suture within a subset of the two or more sutures is kept proportional to each suture's tautness by the suture manifold, and in some embodiments, directly or inversely proportional to the tautness. Thus, the longer the suture, the lower the tautness, or the shorter the suture, the greater the tautness.
Clause 44. The heart valve prosthesis of any of the foregoing embodiments, wherein the length of each suture is kept proportional to a travel displacement by the suture manifold, and in some embodiments, directly proportional to the travel displacement. Thus, the longer the suture, the greater the travel displacement or travel length.
Clause 45. The heart valve prosthesis of any of the foregoing embodiments, the suture manifold further including an inlet, an internal passage, and a plurality of outlets, wherein at least one suture passes through the inlet, the internal passage, and one of the plurality of outlets, such that no two sutures pass through a same outlet of the plurality of outlets.
Clause 46. The heart valve prosthesis of any of the foregoing embodiments, wherein a number of outlets is equal to a number of sutures.
Clause 47. The heart valve prosthesis of any of the foregoing embodiments, the suture manifold further including at least two inlets, wherein at least one suture passes through each inlet.
Clause 48. The heart valve prosthesis of any of the foregoing embodiments, the suture manifold further including at least two internal passages, wherein at least one suture passes through each internal passage.
Clause 49. The heart valve prosthesis of any of the foregoing embodiments, wherein at least one of the internal passages is siloed from another internal passage.
Clause 50. The heart valve prosthesis of any of the foregoing embodiments, wherein the upper end of each suture is coupled into an upper bundle at a point proximal to the inlet.
Clause 51. The heart valve prosthesis of any of the foregoing embodiments, wherein the lower end of each suture is configured to be fastened to a papillary muscle or a ventricular wall of the heart.
Clause 52. The heart valve prosthesis of any of the foregoing embodiments, wherein the lower end of each suture is fastened to a pledget.
Clause 53. The heart valve prosthesis of any of the foregoing embodiments, wherein the lower end of each suture is fastened to a ventricular anchor.
Clause 54. The heart valve prosthesis of any of the foregoing embodiments, wherein the upper end of each suture is configured to be fastened to a mitral annulus, a mitral leaflet, or an atrial wall.
Clause 55. The heart valve prosthesis of any of the foregoing embodiments, further comprising an annuloplasty ring, wherein the suture manifold is fastened to the annuloplasty ring and the annuloplasty ring is fastened to the mitral annulus, the mitral leaflet, or the atrial wall.
Clause 56. The heart valve prosthesis of any of the foregoing embodiments, wherein the suture manifold is fastened to the mitral annulus, the mitral leaflet, or the atrial wall via one or more manifold anchors.
Clause 57. The heart valve prosthesis of any of the foregoing embodiments, further comprising a suture management device, wherein the upper bundle passes through the suture management device.
Clause 58. The heart valve prosthesis of any of the foregoing embodiments, the suture management device further including a locking mechanism and a cutting mechanism.
Clause 59. A delivery catheter comprising an extruded body, a longitudinal slit, a transport element stored within the body, and a filament connected to the transport element and passing through the longitudinal slit.
Clause 60. The delivery catheter of any of the foregoing embodiments, wherein the extruded body is a cylinder or an elliptic cylinder.
Clause 61. The delivery catheter of any of the foregoing embodiments, further comprising a transport element pusher stored within the body and proximal to the transport element, and configured to apply a distal force to the transport element.
Clause 62. The delivery catheter of any of the foregoing embodiments, wherein the filament is connected to a prosthesis.
Clause 63. The delivery catheter of any of the foregoing embodiments, wherein the filament is connected to a second transport element stored within a second delivery catheter.
Clause 64. The delivery catheter of any of the foregoing embodiments, wherein an overlap of the longitudinal slit is between about 0 degrees and 90 degrees.
Clause 65. The delivery catheter of any of the foregoing embodiments, wherein a radial depth of the longitudinal slit is between about 0.2 mm and 2.0 mm.
Clause 66. The delivery catheter of any of the foregoing embodiments, wherein a longitudinal length of the longitudinal slit is between about 1 mm to about 500 mm.
Clause 67. The delivery catheter of any of the foregoing embodiments, wherein the longitudinal slit terminates at a distal end of the body.
Clause 68. A prosthesis delivery system comprising: a delivery sheath; a prosthesis device housed within the delivery sheath; and a plurality of delivery catheters housed within the delivery sheath including a first delivery catheter, each delivery catheter having a longitudinal slit and housing a transport element and a transport element pusher.
Clause 69. The prosthesis delivery system of any of the foregoing embodiments, wherein a filament runs from a first transport element housed within the first delivery catheter and through a first longitudinal slit of the first delivery catheter.
Clause 70. The prosthesis delivery system of any of the foregoing embodiments, further comprising at least a second delivery catheter, wherein the filament runs from the first transport element, through the first longitudinal slit of the first delivery catheter, through a second longitudinal slit of the second delivery catheter, and connects to a second transport element housed within the second delivery catheter.
Clause 71. The prosthesis delivery system of any of the foregoing embodiments, wherein the filament runs from the first transport element, through the first longitudinal slit of the first delivery catheter, to the prosthesis device.
Clause 72. The prosthesis delivery system of any of the foregoing embodiments, wherein the filament connects one or more transport elements.
Clause 73. The prosthesis delivery system of any of the foregoing embodiments, wherein an outer diameter of the delivery sheath is between about 4 mm and 15 mm.
Clause 74. The prosthesis delivery system of any of the foregoing embodiments, wherein an outer diameter of each delivery catheter is between about 0.5 mm and 5 mm.
Clause 75. The prosthesis delivery system of any of the foregoing embodiments, wherein two or more of the plurality of catheters have the same diameter and share the function within the system.
Clause 76. The prosthesis delivery system of any of the foregoing embodiments, wherein one of the plurality of catheters serves a different function than another of the plurality of catheters; and is arranged coaxial to the delivery sheath; and/or has a larger diameter than the other of the plurality of catheters.
Clause 77. The prosthesis delivery system of any of the foregoing embodiments, wherein one or more of the plurality of catheters is arranged radially symmetrically about a central axis of the delivery sheath.
Clause 78. The prosthesis delivery system of any of the foregoing embodiments, the prosthesis device further comprising: a suture manifold; and a suture net including two or more sutures; wherein a lower end of the sutures passes through the first longitudinal slit of the first delivery catheter and an upper end of the sutures passes through a second longitudinal slit of a second delivery catheter.
Clause 79. The prosthesis delivery system of any of the foregoing embodiments, the prosthesis device further comprising a suture manifold including a manifold anchor, wherein the manifold anchor is configured to be fixed to a target anatomy by the transport element.
Clause 80. The prosthesis delivery system of any of the foregoing embodiments, wherein the delivery sheath is sufficiently flexible and steerable to pass through an atrial septum or aortic arch.
Clause 81. A method of surgically repairing a heart valve comprising the steps of: attaching a ventricle fastener to a ventricle of a heart; attaching a suture manifold to an atrium of a heart, or an implanted annuloplasty ring; and adjusting a length of two or more sutures concurrently and equally, including: retracting a bundle comprised of an upper end of each of the two or more sutures; locking the length of the two or more sutures concurrently; and removing an excess from the two or more sutures concurrently; wherein the two or more sutures connect at a lower end to the ventricle fastener and connect at an upper end to the suture manifold.
Clause 82. The method of any of the foregoing embodiments, wherein the step of attaching the ventricle fastener further comprises: suturing a bundle comprising a lower end of each of the two or more sutures to a pledget and to the heart.
Clause 83. The method of any of the foregoing embodiments, wherein the step of attaching the ventricle fastener further comprises: attaching the ventricle fastener to a papillary muscle.
Clause 84. The method of any of the foregoing embodiments, wherein the step of attaching the suture manifold further comprises: suturing an annuloplasty ring to the heart; and attaching the suture manifold to the annuloplasty ring.
Clause 85. The method of any of the foregoing embodiments, wherein the step of attaching the suture manifold further comprises: attaching the annuloplasty ring to an atrioventricular valve annulus.
Clause 86. The method of any of the foregoing embodiments, wherein the step of attaching the suture manifold further comprises directly attaching the suture manifold to a mitral annulus, a mitral leaflet, or an atrial wall via one or more manifold anchors.
Clause 87. The method of any of the foregoing embodiments, wherein the one or more sutures travel from the ventricle fastener, through a plurality of outlets of the suture manifold, an internal passage of the suture manifold, and an inlet of the suture manifold. Clause 88. The method of any of the foregoing embodiments, further comprising: adjusting a length of two or more additional sutures concurrently and equally, including retracting a second bundle comprised of an upper end of each of the two or more additional sutures.
Clause 89. The method of any of the foregoing embodiments, wherein the two or more sutures includes a first set of sutures and a second set of sutures; wherein a tautness of the first set of sutures differs from a tautness of the second set of sutures.
Clause 90. The method of any of the foregoing embodiments, wherein the step of locking further comprises crimping a sleeve onto the upper end of each suture.
Clause 91. A method of repairing a heart valve via transcatheter implant comprising the steps of: advancing a delivery sheath through an atrioventricular valve and to a ventricle of the heart, the delivery sheath containing a ventricle fastener, a suture manifold, an atrial fastener, a suture management device, two or more sutures, and a plurality of catheters; deploying the ventricle fastener at a ventricle fastening site; withdrawing the delivery sheath to an atrium of the heart; deploying the atrial fastener at the atrium fastening site; adjusting a length of the two or more sutures concurrently; locking the two or more sutures concurrently; removing an excess from the two or more sutures concurrently; and withdrawing the delivery sheath.
Clause 92. The method of any of the foregoing embodiments, wherein the procedure is a mitral valve repair and the delivery sheath is advanced through a femoral vein, jugular vein, femoral artery, or via a transapical approach.
Clause 93. The method of any of the foregoing embodiments, wherein the procedure is a tricuspid valve repair and the delivery sheath is advanced through a femoral vein or a jugular vein.
Clause 94. The method of any of the foregoing embodiments, wherein the plurality of catheters includes a ventricle fastener catheter comprising a longitudinal slit and containing the ventricle fastener and a ventricle fastener pusher, wherein the step of deploying the ventricle fastener further comprises: distally advancing the ventricle fastener pusher such that the ventricle fastener exits the ventricle fastener catheter; and withdrawing the ventricle fastener catheter.
Clause 95. The method of any of the foregoing embodiments, wherein the two or more sutures pass through the longitudinal slit of the ventricle fastener catheter and through the suture manifold.
Clause 96. The method of any of the foregoing embodiments, wherein the plurality of catheters includes an atrial fastener catheter, the atrial fastener catheter comprising a longitudinal slit and containing the atrial fastener and an atrial fastener pusher, wherein the step of deploying the atrial fastener further comprises: distally advancing the atrial fastener pusher such that the atrial fastener exits the atrial fastener catheter; and withdrawing the atrial fastener catheter.
Clause 97. The method of any of the foregoing embodiments, wherein the step of deploying the atrial fastener further comprises fixing a suture manifold anchor to the atrium fastening site.
Clause 98. The method of any of the foregoing embodiments, further comprising tensioning the atrial fastener to draw the manifold into its final position.
Clause 99. The method of any of the foregoing embodiments, wherein the step of deploying the atrial fastener(s) further comprises: repeating the steps of any of the foregoing embodiments for one or more atrial fastener catheters, each comprising a longitudinal slit and containing a respective atrial fastener and a respective atrial fastener pusher.
Clause 100. The method of any of the foregoing embodiments, wherein the delivery sheath further contains two or more sutures, each suture connecting at a lower end to the ventricle fastener, each suture passing through an outlet of the suture manifold, each suture passing through an inlet of the suture manifold, and each suture connecting at an upper end to the atrial fastener.
Clause 101. The method of any of the foregoing embodiments, wherein the step of adjusting the length further comprises: proximally withdrawing an upper bundle, wherein the upper bundle is formed by connecting each suture at the upper end proximal to the inlet of the suture manifold.
Clause 102. The method of any of the foregoing embodiments, wherein the delivery sheath further comprises and houses a tightening catheter, the tightening catheter including and housing a tightening transport element connected to the upper bundle, wherein the step of adjusting the length of the two or more sutures further comprises proximally retracting or distally advancing the tightening transport element.
Clause 103. The method of any of the foregoing embodiments, wherein the delivery sheath further comprises and houses a suture management catheter, the suture management catheter including and housing a suture management transport element, wherein the step of locking the two or more sutures and the step of removing an excess from the two or more sutures further comprises distally advancing the suture management transport element to the upper bundle and actuating the suture management transport element.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to about 10 percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed:

1. A suture manifold comprising a body, a first inlet, a first internal passage, and a plurality of outlets, wherein the first inlet is connected to at least one of the plurality of outlets through the first internal passage.

2. The suture manifold of claim 1, further comprising a second inlet and a second internal passage, wherein the second inlet is connected to at least one of the plurality of outlets through the second internal passage.

3. The suture manifold of claim 2, wherein the first internal passage and the second internal passage are siloed from one another.

4. The suture manifold of claim 1, further comprising a plurality of inlets and a plurality of internal passages, wherein each internal passage connects one of the inlets to at least one of the plurality of outlets, and wherein at least one internal passage of the plurality is siloed from at least one other internal passage of the plurality of internal passages.

5. The suture manifold of claim 3, wherein the first internal passage and the second internal passage are siloed by a frontal internal wall or a transverse internal wall.

6. The suture manifold of claim 3, wherein the first internal passage and the second internal passage are siloed by a sagittal internal wall.

7. The suture manifold of claim 1, wherein the body is cylindrical in shape.

8. The suture manifold of claim 1, wherein one or more dimensions of the suture manifold are configured to correspond to a number of sutures passing through the body.

9. The suture manifold of claim 1, wherein the body has a longitudinal length between about 10 mm and 60 mm and a diameter between about 1 mm and 20 mm.

10. An adjustable suture net comprising at least two vertical sutures, each vertical suture being fixed at an upper anchor point and a lower anchor point, and at least one horizontal thread tethering one or more vertical sutures, wherein a length of each vertical suture between the upper anchor point and the lower anchor point is adjustable; and wherein the length of each vertical suture controls a tautness of each vertical suture.

11. The adjustable suture net of claim 10, wherein the at least one horizontal thread comprises two or more horizontal threads spaced along a vertical axis.

12. The adjustable suture net of claim 10, further comprising an upper bundle tethering together an upper end of each vertical suture and a lower bundle tethering together a lower end of each vertical suture.

13. The adjustable suture net of claim 10, further comprising a fastening device configured to adjust the length of each vertical suture.

14. A heart valve prosthesis comprising:
two or more vertical sutures, each suture having an upper end configured to be fastened to an atrium of a heart and a lower end configured to be fastened to a ventricle of the heart; and
a suture manifold directing a path of each of the two or more vertical sutures;
wherein the suture manifold controls a length of each suture.

15. The heart valve prosthesis of claim 14, wherein the lower end of each suture is coupled into a lower bundle before being fastened to the ventricle of the heart.

16. The heart valve prosthesis of claim 14, further comprising a net located below the suture manifold, formed by the two or more sutures and at least one horizontal thread connecting each suture.

17. The heart valve prosthesis of claim 14, wherein the length of each suture within a subset of the two or more sutures its kept inversely proportional to each suture's tautness by the suture manifold.

18. The heart valve prosthesis of claim 14, the suture manifold further including an inlet, an internal passage, and a plurality of outlets, wherein at least one suture passes through the inlet, the internal passage, and one of the plurality of outlets, such that no two sutures pass through a same outlet of the plurality of outlets.

19. The heart valve prosthesis of claim 18, wherein a number of outlets is equal to a number of sutures.

20. The heart valve prosthesis of claim 18, the suture manifold further including at least two internal passages, wherein at least one suture passes through each internal passage.

21. The heart valve prosthesis of claim 14, wherein the lower end of each suture is fastened to a pledget, or a ventricular anchor.

22. The heart valve prosthesis of claim 14, further comprising an annuloplasty ring, wherein the suture manifold is fastened to the annuloplasty ring and the annuloplasty ring is fastened to the mitral annulus, the mitral leaflet, or the atrial wall.

23. A prosthesis delivery system comprising:
a delivery sheath;
a prosthesis device housed within the delivery sheath; and
a plurality of delivery catheters housed within the delivery sheath including a first delivery catheter, each delivery catheter having a longitudinal slit and housing a transport element and a transport element pusher.

24. The prosthesis delivery system of claim 23, wherein a filament runs from a first transport element housed within the first delivery catheter and through a first longitudinal slit of the first delivery catheter.

25. The prosthesis delivery system of claim 24, further comprising at least a second delivery catheter, wherein the filament runs from the first transport element, through the first longitudinal slit of the first delivery catheter, through a second longitudinal slit of the second delivery catheter, and connects to a second transport element housed within the second delivery catheter.

26. The prosthesis delivery system of claim 23, the prosthesis device further comprising:
a suture manifold; and
a suture net including two or more sutures;
wherein a lower end of the sutures passes through the first longitudinal slit of the first delivery catheter and an upper end of the sutures passes through a second longitudinal slit of a second delivery catheter.
